(19) United States
(12) Patent Application Publication
(10) Pub. No.: US 2025/0269106 A1
(43) Pub. Date: Aug. 28, 2025

(54) SYSTEM, METHOD AND COMPUTER PROGRAM FOR GUIDED WORKFLOW FEATURES FOR OPERATING A FLUID INJECTOR SYSTEM

(71) Applicant: Bayer Healthcare LLC, Whippany, NJ (US)

(72) Inventors: Mikayla FERCHAW, Pittsburgh, PA (US); Kyle TANCZOS, Pittsburgh, PA (US); Sydney SANDIDGE, Pittsburgh, PA (US); Patrick O'ROURKE, New Middletown, OH (US); Randy LEE, Pittsburgh, PA (US); Michael SPOHN, Fenelton, PA (US); Kevin COWAN, Allison Park, PA (US); Christopher SCUTT, Murrysville, PA (US); John HAURY, Sewickley, PA (US); David BERRY, Kittanning, PA (US); Irina SAMKOVA, Saint Petersburg, FL (US)

(21) Appl. No.: 18/858,568

(22) PCT Filed: May 3, 2023
(86) PCT No.: PCT/US2023/020849

§ 371 Date: Oct. 21, 2024

Related U.S. Application Data
(60) Provisional application No. 63/338,333, filed on May 4, 2022.

(51) Int. Cl. A61M 5/00; G16H 70/20

(52) U.S. Cl. CPC A61M 5/007; G16H 70/20; A61M 2205/502; A61M 2205/6072; A61M 2205/702

(57) ABSTRACT

Methods for providing one or more guided workflow features with regard to a fluid injector system, may include receiving data associated with the fluid injector system, displaying a first graphical user interface (GUI) screen associated with a guided workflow for operation of the fluid injector system on one or more display devices based on the data associated with one or more components of the fluid injector system, receiving a first user input, displaying a second GUI screen associated with the guided workflow on the one or more display devices based on receiving the first user input, executing a first operation of the fluid injector system, and receiving a second user input, where the second GUI screen provides information that is sequential to the first GUI screen with regard to the guided workflow for operation of the fluid injector system. Systems and computer program products are also disclosed.

### CROSS-REFERENCE TO RELATED APPLICATION

This application claims the benefit of U.S. Provisional Application No. 63/338,333, filed May 4, 2022, the disclosure of which is incorporated by reference in its entirety.

### BACKGROUND

### 1. Field

This disclosure relates generally to systems, devices, products, apparatus, and methods for operating medical fluid injector systems and optimizing workflow features and algorithms for injection procedures for medical fluid injector systems.

### 2. Technical Considerations

An injection device, such as a fluid injection device, may be used by a medical practitioner in a medical diagnostic procedure and/or a medical therapeutic procedure. For example, the medical practitioner may use the fluid injection device to inject a patient with one or more medical fluids. The fluid injection device may be used for pressurized injection of a medical fluid, such as a radiological contrast material (e.g., a contrast agent, a radiocontrast agent, contrast media, etc.), and/or a flushing agent, such as saline, in medical imaging procedures, such as angiography, computed tomography (CT), ultrasound, magnetic resonance imaging (MRI), and positron emission tomography (PET). In some instances, the fluid injection device may be designed to deliver a preset amount of one or more medical fluids at a preset pressure and/or flow rate to a patient.

Generally, fluid injector devices may have a drive member, such as a piston, that connects to a syringe, for example via connection with a syringe plunger. The syringe may include a barrel that is rigid with the syringe plunger being slidably disposed within the barrel. The drive member may drive the syringe plunger and/or the rolling diaphragm/proximal end wall in a proximal and/or distal direction relative to a longitudinal axis of the barrel to draw fluid into or deliver the fluid from a syringe barrel.

Conventional fluid injectors systems may include a series of steps or processes for setting up a fluid injection procedure, conducting a fluid injection procedure, preparing the system for a subsequent fluid injection procedure. The series of steps or processes may be long and complex and the potential for skipping or inadvertently missing one or more steps may result in sub-optimal images, potential harm to a patient, and/or require repeating the fluid injection and imaging procedure. Guided workflow procedures that guide a user through an injection procedure, as described herein, may improve injection procedures and limit mistakes, resulting in better image quality and limit errors or potential harm to a patient.

### SUMMARY

Accordingly, provided are systems, methods, and computer program products for providing one or more guided workflow features with regard to a fluid injector system that improve the efficiency and accuracy of operation of the fluid injector system during a fluid injection procedure and for preparing the fluid injector system for a subsequent injection procedure.

According to embodiments, a method for providing one or more guided workflow features with regard to a fluid injector system, includes receiving, with at least one processor, data associated with the fluid injector system; displaying, with the at least one processor, a first graphical user interface (GUI) screen associated with a guided workflow for operation of the fluid injector system on one or more display devices based on the data associated with one or more components of the fluid injector system; receiving, with the at least one processor, a first user input via a first graphical element of the first GUI screen or an input device; displaying, with the at least one processor, a second GUI screen associated with the guided workflow for operation of the fluid injector system on the one or more display devices based on receiving the first user input; executing, with the at least one processor, a first operation of the fluid injector system based on the first user input; and receiving, with the at least one processor, a second user input via a second graphical element of the second GUI screen or the input device; wherein the second GUI screen provides information that is sequential to the first GUI screen with regard to the guided workflow for operation of the fluid injector system.

According to embodiments, the method includes executing a second operation of the fluid injector system based on the second user input.

According to embodiments, the method, wherein displaying the first GUI screen on the one or more display devices, includes displaying the first GUI screen on one or more display devices of the fluid injector system.

According to embodiments, the method, wherein displaying the first GUI screen on the one or more display devices, incudes displaying at least a first portion of the first GUI screen on a first display device of the fluid injector system, and displaying at least a second portion of the first GUI screen on a second display device that is independent of the fluid injector system.

According to embodiments, the method, wherein displaying the second GUI screen on the one or more display devices, includes displaying at least a first portion of the second GUI screen on a first display device of the fluid injector system based on receiving the first user input via the graphical element of the first GUI screen or the input device, and displaying at least a second portion of the second GUI screen on a second display device that is independent of the fluid injector system based on receiving the first user input via the graphical element of the first GUI screen or the input device.

According to embodiments, the method, wherein displaying the first GUI screen on the one or more display devices, includes displaying the first GUI screen on a display device of a workstation device that is independent of the fluid injector system.

According to embodiments, the method, wherein receiving data associated with the fluid injector system, includes receiving data associated with one or more disposable components of the fluid injector system from a reader device that reads at least one barcode associated with the one or more disposable components of the fluid injector system. The method further includes displaying on the one or more display devices, a first GUI screen associated with a guided workflow for disposal and recycling of one or more components used during a fluid injection procedure of the fluid injector system based on the data associated with the one or more disposable components of the fluid injector system, receiving a third user input via a graphical element of the first GUI screen associated with a guided workflow for recycling of one or more components used during the fluid injection procedure of the fluid injector system or the input device, wherein the third user input comprises an indication that the one or more disposable components of the fluid injector system are to be recycled, displaying on the one or more display devices, a second GUI screen associated with the guided workflow for disposal and recycling of the one or more components based on the receiving the third user input, wherein the second GUI screen comprises an instruction with regard to a procedure for recycling the one or more disposable components of the fluid injector system, and displaying on the one or more display devices, a third GUI screen associated with the guided workflow for disposal and recycling of the one or more components, wherein the second GUI screen comprises data associated with a sustainability score indicating an environmental sustainability of a user.

According to embodiments, a system for providing one or more guided workflow features on a fluid injector system includes at least one processor programmed or configured to receive data associated with the fluid injector system, display a first graphical user interface (GUI) screen associated with a guided workflow for operation of the fluid injector system on one or more display devices based on the data associated with one or more components of the fluid injector system, receive a first user input via a first graphical element of the first GUI screen or an input device, display a second GUI screen associated with the guided workflow for operation of the fluid injector system on the one or more display devices based on receiving the first user input, execute a first operation of the fluid injector system based on the first user input, and receive a second user input via a second graphical element of the second GUI screen or the input device, and the second GUI screen provides information that is sequential to the first GUI screen with regard to the guided workflow for operation of the fluid injector system.

According to embodiments, the at least one processor is further programmed or configured to execute a second operation of the fluid injector system based on the second user input.

According to embodiments, when displaying the first GUI screen on the one or more display devices, the at least one processor is programmed or configured to display the first GUI screen on one or more display devices of the fluid injector system.

According to embodiments, when displaying the first GUI screen on the one or more display devices, the at least one processor is programmed or configured to display at least a first portion of the first GUI screen on a first display device of the fluid injector system, and display at least a second portion of the first GUI screen on a second display device that is independent of the fluid injector system.

According to embodiments, the at least one processor is further programmed or configured to display at least a first portion of the second GUI screen on a first display device of the fluid injector system based on receiving a user input via a graphical element of the first GUI screen or the input device, and display at least a second portion of the second GUI screen on a second display device that is independent of the fluid injector system based on receiving the user input via the graphical element of the first GUI screen or the input device.

According to embodiments, when displaying the first GUI screen on the one or more display devices, the at least one processor is programmed or configured to display the first GUI screen on a display device of a workstation device that is independent of the fluid injector system.

According to embodiments, when receiving data associated with the fluid injector system, the at least one processor is programmed or configured to receive data associated with one or more disposable components of the fluid injector system from a reader device that reads at least one barcode associated with the one or more disposable components of the fluid injector system. The at least one processor is further programmed or configured to display on the one or more display devices, a first GUI screen associated with a guided workflow for disposal and recycling of one or more components used during a fluid injection procedure of the fluid injector system based on the data associated with the one or more disposable components of the fluid injector system; receive a third user input via a graphical element of the first GUI screen associated with a guided workflow for recycling of one or more components used during the fluid injection procedure of the fluid injector system or the input device, wherein the third user input includes an indication that the one or more disposable components of the fluid injector system are to be recycled; display on the one or more display devices, a second GUI screen associated with the guided workflow for disposal and recycling of the one or more components based on the receiving the third user input, wherein the second GUI screen comprises an instruction with regard to a procedure for recycling the one or more disposable components of the fluid injector system; and display on the one or more display devices, a third GUI screen associated with the guided workflow for disposal and recycling of the one or more components, wherein the second GUI screen comprises data associated with a sustainability score indicating an environmental sustainability of a user.

According to embodiments, a computer program product for providing one or more guided workflow features with regard to a fluid injector system includes at least one non-transitory computer-readable medium including one or more instructions that, when executed by at least one processor, cause the at least one processor to receive data associated with the fluid injector system, display a first graphical user interface (GUI) screen associated with a guided workflow for operation of the fluid injector system on one or more display devices based on the data associated with one or more components of the fluid injector system, receive a first user input via a first graphical element of the first GUI screen or an input device, display a second GUI screen associated with the guided workflow for operation of the fluid injector system on the one or more display devices based on receiving the first user input, execute a first operation of the fluid injector system based on the first user input, and receive a second user input via a second graphical element of the second GUI screen or the input device, and the second GUI screen provides information that is sequential to the first GUI screen with regard to the guided workflow for operation of the fluid injector system.

According to embodiments, the one or more instructions further cause the at least one processor to execute a second operation of the fluid injector system based on the second user input.

According to embodiments, the one or more instructions that cause the at least one processor to display the first GUI screen on the one or more display devices, cause the at least one processor to display the first GUI screen on one or more display devices of a fluid injector system.

According to embodiments, the one or more instructions further cause the at least one processor to display at least a first portion of the first GUI screen on a first display device of a fluid injector system, and display at least a second portion of the first GUI screen on a second display device that is independent of the fluid injector system.

According to embodiments, the one or more instructions that cause the at least one processor to display the first GUI screen on the one or more display devices, cause the at least one processor to display at least a first portion of the second GUI screen on a first display device of the fluid injector system based on receiving the user input via the graphical element of the first GUI screen or the input device, and display at least a second portion of the second GUI screen on a second display device that is independent of the fluid injector system based on receiving the user input via the graphical element of the first GUI screen or the input device.

According to embodiments, the one or more instructions that cause the at least one processor to receive data associated with the fluid injector system, cause the at least one processor to receive data associated with one or more components of the fluid injector system from a reader device that read a barcode associated with the one or more components of the fluid injector system. The one or more instructions further cause the at least one processor to display on the one or more display devices, a first GUI screen associated with a guided workflow for disposal and recycling of one or more components used during a fluid injection procedure of the fluid injector system based on the data associated with the one or more disposable components of the fluid injector system, receive a third user input via a graphical element of the first GUI screen associated with a guided workflow for recycling of one or more components used during the fluid injection procedure of the fluid injector system or the input device, wherein the third user input comprises an indication that the one or more disposable components of the fluid injector system are to be recycled, display on the one or more display devices, a second GUI screen associated with the guided workflow for disposal and recycling of the one or more components based on the receiving the third user input, wherein the second GUI screen comprises an instruction with regard to a procedure for recycling the one or more disposable components of the fluid injector system, and display on the one or more display devices, a third GUI screen associated with the guided workflow for disposal and recycling of the one or more components, and the second GUI screen comprises data associated with a sustainability score indicating an environmental sustainability of a user.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present disclosure.

### BRIEF DESCRIPTION OF THE DRAWINGS

Additional advantages and details of non-limiting embodiments or aspects are explained in greater detail below with reference to the exemplary embodiments that are illustrated in the accompanying schematic figures, in which:

FIG. 1A is a diagram of a non-limiting embodiment of an environment in which systems, devices, products, apparatus, and/or methods, described herein, may be implemented, according to the principles of the present disclosure;

FIG. 1B is a diagram of a non-limiting embodiment of a system for providing one or more guided workflow features with regard to a fluid injector system;

FIG. 2 is a diagram of a non-limiting embodiment of components of one or more systems or one or more devices of FIGS. 1A and 1B;

FIG. 3 is a flowchart of a non-limiting embodiment of a process for providing one or more guided workflow features with regard to a fluid injector system;

FIG. 4 is an illustration of a non-limiting embodiment of an introductory graphical user interface (GUI) screen of a guided workflow for operation of a fluid injector system;

FIG. 5 is an illustration of a non-limiting embodiment of a GUI screen of a guided workflow for setting a configuration of one or more disposable components of a fluid injection system;

FIGS. 6A-6B are illustrations of non-limiting embodiments of GUI screens of a guided workflow for selection of one or more disposable components of a fluid injection system;

FIGS. 7A-7G are illustrations of non-limiting embodiments of GUI screens of a guided workflow for setup of one or more fluids to be used with a fluid injector system during a fluid injection procedure;

FIGS. 8A-8G are illustrations of non-limiting embodiments of GUI screens of a guided workflow for filling of one or more syringes with one or more fluids to be used during a fluid injection procedure;

FIGS. 9A-9B are illustrations of non-limiting embodiments of GUI screens of a guided workflow for connecting one or more tubing set components of a fluid injection system;

FIGS. 10A-10J are illustrations of non-limiting embodiments of GUI screens of a guided workflow for allowing a fluid injector system to manually purge or automatically purge air from one or more components of the fluid injector system are illustrated;

FIG. 11 is an illustration of a non-limiting embodiment of a GUI screen of a guided workflow for notification of a hazard;

FIG. 12 is an illustration of a non-limiting embodiment of a GUI screen of a guided workflow for notification regarding a connection of one or more tubing components of a fluid injector system;

FIGS. 13A-13D are illustrations of a non-limiting embodiments of GUI screens of a guided workflow for setup of one or more disposable components of a fluid injector system to be used during a fluid injection procedure;

FIG. 14 is an illustration of a non-limiting embodiment of a GUI screen providing a status indication of steps of a guided workflow for operation of a fluid injector system;

FIG. 15 is an illustration of a non-limiting embodiment of a GUI screen providing information regarding one or more fluid injection protocols that are to be used during a fluid injection procedure;

FIG. 16 is an illustration of a non-limiting embodiment of a GUI screen providing summary information regarding one or more disposable components of a fluid injector system to be used during a fluid injection procedure;

FIGS. 17A-17E are illustrations of non-limiting embodiments of GUI screens of a guided workflow for disassembly of one or more components of a fluid injector system; and

FIG. 18 is a diagram of a non-limiting embodiment of an environment including a workstation device and a fluid injector system.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the present disclosure as it is oriented in the drawing figures. However, it is to be understood that the present disclosure may assume various alternative variations and step sequences, except where expressly specified to the contrary. It is also to be understood that the specific devices and processes illustrated in the attached drawings, and described in the following specification, are simply exemplary embodiments of the present disclosure. Hence, specific dimensions and other physical characteristics related to the embodiments or aspects of the embodiments disclosed herein are not to be considered as limiting unless otherwise indicated.

Spatial or directional terms, such as “left”, “right”, “inner”, “outer”, “above”, “below”, and the like, relate to the invention as shown in the drawing figures and are not to be considered as limiting as the invention can assume various alternative orientations.

All numbers used in the specification and claims are to be understood as being modified in all instances by the term “about”. The term “about” is meant to include plus or minus twenty-five percent of the stated value, such as plus or minus ten percent of the stated value. However, this should not be considered as limiting to any analysis of the values under the doctrine of equivalents. The terms “first”, “second”, and the like are not intended to refer to any particular order or chronology, but refer to different conditions, properties, or elements unless otherwise specified.

The term “at least” is synonymous with “greater than or equal to”. The term “not greater than” is synonymous with “less than or equal to”. As used herein, “at least one of” is synonymous with “one or more of”. For example, the phrase “at least one of A, B, and C” means any one of A, B, or C, or any combination of any two or more of A, B, or C. For example, “at least one of A, B, and C” includes A alone; or B alone; or C alone; or A and B; or A and C; or B and C; or all of A, B, and C. The term “includes” is synonymous with “comprises”. No aspect, component, element, structure, act, step, function, instruction, and/or the like used herein should be construed as critical or essential unless explicitly described as such. As used in the specification and the claims, the singular form of “a,” “an,” and “the” include plural referents unless the context clearly dictates otherwise. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

When used in relation to a medical device, such as a syringe, the term “proximal” refers to a portion of a syringe nearest a piston element for engaging with an end wall of the syringe and delivering fluid from a syringe. When used in relation to a fluid path, the term “proximal” refers to a portion of the fluid path nearest to an injector system when the fluid path is connecting with the injector system. When used in relation to a syringe, the term “distal” refers to a portion of a syringe nearest to a delivery nozzle. When used in relation to a fluid path, the term “distal” refers to a portion of the fluid path nearest to a patient when the fluid path is connected with an injector system. The term “radial” refers to a direction in a cross-sectional plane normal to a longitudinal axis of a syringe extending between proximal and distal ends. The term “circumferential” refers to a direction around an inner or outer surface of a sidewall of a syringe. The term “axial” refers to a direction along a longitudinal axis of the syringe extending between the proximal and distal ends.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In embodiments, a message may refer to a network packet (e.g., a data packet and/or the like) that includes data. It will be appreciated that numerous other arrangements are possible.

As used herein, the term “system” may refer to one or more computing devices or combinations of computing devices such as, but not limited to, processors, servers, client devices, software applications, and/or other like components. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

In some instances, a fluid injector system may be a part of a group of devices that are used in a medical treatment facility during treatment of patients, and/or in a medical imaging facility. Individuals, such as medical care providers (e.g., personnel that operate the fluid injector system and associated medical imaging systems), may manually configure one or more components of a fluid injector system prior to, during, and/or following a fluid injection procedure. However, a fluid injector system (e.g., a fluid injector device of a fluid injector system) may require a series of processes for setting up a fluid injection procedure, conducting a fluid injection procedure, and/or preparing the fluid injector device for a subsequent fluid injection procedure. The series of processes may be long and complex and the potential for skipping, making a mistake during performance, or inadvertently failing to perform one or more steps of a process may result in sub-optimal results (e.g., images) from a fluid injection and imaging procedure, potential harm to a patient, and/or require repeating the fluid injection and imaging procedure.

The described systems, methods, and computer program products provide one or more guided workflow features with regard to a fluid injector system. In embodiments, an injector management system may receive data associated with a fluid injector system, obtain a first graphical user interface (GUI) screen associated with a guided workflow for operation of the fluid injector system to be displayed on one or more display devices based on the data associated with one or more components of the fluid injector system; display the first GUI screen on one or more display devices, receive a user input via a graphical element of the first GUI screen, obtain a second GUI screen associated with the guided workflow for operation of the fluid injector system to be displayed on the one or more display devices, and display the second GUI screen on the one or more display devices, where the second GUI screen provides information that is sequential to the first GUI screen with regard to the guided workflow for operation of the fluid injector system. Injector management system may execute an operation of the fluid injector system based on a user input received via a graphical element of the first GUI screen.

In embodiments, when displaying the first GUI screen on the one or more display devices, the injector management system may display the first GUI screen on one or more display devices of a fluid injector system. When displaying the first GUI screen on the one or more display devices, the injector management system may display at least a first portion of the first GUI screen on a first display device of a fluid injector system and display at least a second portion of the first GUI screen on a second display device of the fluid injector system. In other embodiments, when displaying the first GUI screen on the one or more display devices, the injector management system may display at least a first portion of the second GUI screen on a first display device of a fluid injector system based on receiving the user input received via the graphical element of the first GUI screen and display at least a second portion of the second GUI screen on a second display device independent of the fluid injector system based on receiving the user input received via the graphical element of the first GUI screen.

In embodiments, when displaying the first GUI screen on the one or more display devices, the injector management system may display at least a portion of the first GUI screen on a display device of a workstation device that is independent of a fluid injector system. When receiving data associated with the fluid injector system, the injector management system may receive data associated with one or more components of the fluid injector system from a reader device that reads (e.g., scans) a barcode associated with the one or more components of the fluid injector system.

In this way, fluid injector systems of the present disclosure provide one or more guided workflow features that may be used to guide a user through a fluid injection procedure, which may improve the results of fluid injection procedures, speed up one or more processes associated with the fluid injection procedure, and/or limit mistakes, resulting in better image quality and limit errors and/or potential harm to a patient. The guided workflow features may automatically lead a user through a series of steps associated with accurate and safe preparation of a fluid injector system to conduct at least a portion of a fluid injection procedure, such as at least one of injection programming, equipment setup procedures, purging/priming procedures, injection procedures, and/or teardown and disposal/recycling of one or more components of the fluid injector system.

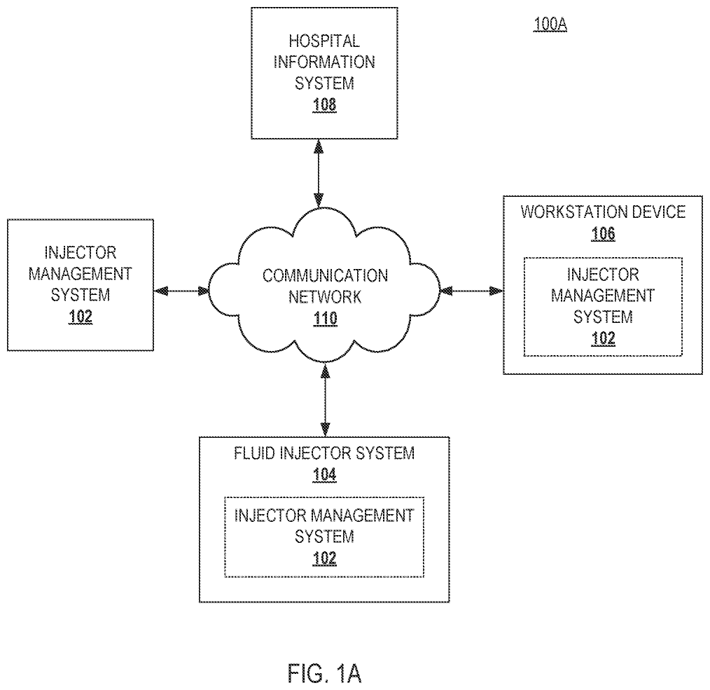

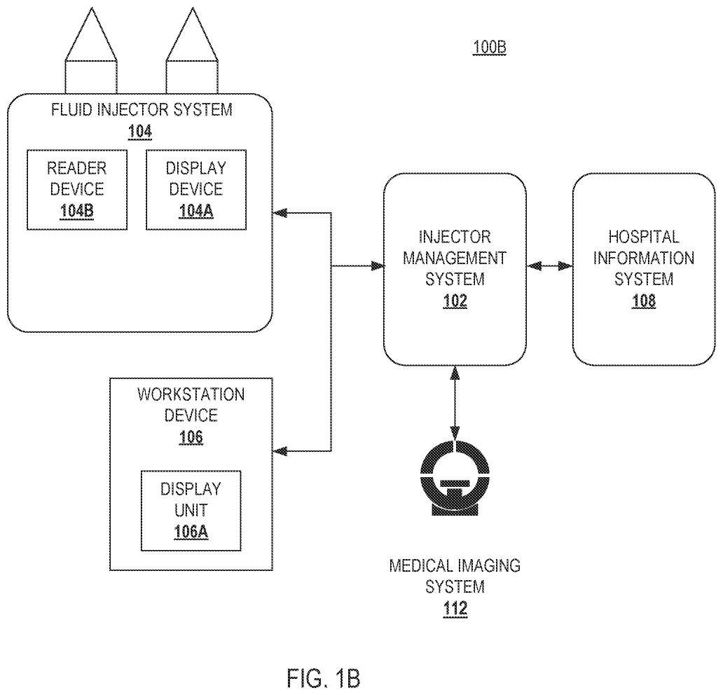

Referring now to FIG. 1A, FIG. 1A is a diagram of a non-limiting embodiment of environment 100A in which devices, systems, methods, and/or computer program products, described herein, may be implemented. As shown in FIG. 1A, environment 100A includes injector management system 102, fluid injector system 104, workstation device 106, hospital information system 108, and communication network 110. Injector management system 102, fluid injector system 104, workstation device 106, and/or hospital information system 108, may interconnect via wired connections, wireless connections, or a combination of wired and wireless connections.

In embodiments, injector management system 102 may include one or more devices capable of being in communication with one or more of fluid injector system 104, workstation device 106, and/or hospital information system 108, via communication network 110. For example, injector management system 102 may include one or more computing devices, such as one or more computers, one or more controllers, one or more servers, one or more desktop computers, one or more mobile devices, and/or the like. Injector management system 102 may include one or more software and/or hardware applications that perform a set of functionalities on an external application programming interface (API) that allows injector management system 102 to send data to an external system associated with the external API and to receive data from the external system associated with the external API. In some embodiments, the application may be supported by an application associated with fluid injector system 104 that allows injector management system 102, e.g., a control room display, to be the only one device that controls fluid injector system 104 and, in such an example, injector management system 102 may provide an authentication function. In embodiments, injector management system 102 may be a component of fluid injector system 104 and/or workstation device 106.

In some embodiments, fluid injector system 104 may include one or more devices capable of being in communication with injector management system 102, workstation device 106, and/or hospital information system 108 via communication network 110. Fluid injector system 104 may include one or more fluid injection devices configured to administer a contrast agent to a patient, and/or administer an aqueous flushing fluid, such as saline, to a patient before, during, and/or after administering the contrast fluid. In embodiments, fluid injector system 104 may include one or more exemplary fluid injection devices that are disclosed in: U.S. Pat. Nos. 6,643,537; 7,094,216; 7,556,619; 8,337,456; 8,147,464; 8,540,698, 9,173,995, 10,507,319, and PCT International Application Publication Nos. WO 2022/119837 and WO 2022/182935, the disclosures of each of which are incorporated by reference herein in their entireties. In embodiments, fluid injector system 104 that may utilize the guided workflow processes described herein may include the Bayer MEDRAD® Stellant CT Injection System, the Bayer MEDRAD® Stellant FLEX CT Injection System, the Bayer MEDRAD® MRXperion MR Injection System, the Bayer MEDRAD® Mark 7 Arterion Injection System, the Bayer MEDRAD® Intego PET Infusion System, the Bayer MEDRAD® Centargo CT Injection System, or other Bayer sourced injector systems used for angiography and computed tomography all of which are provided by Bayer Healthcare LLC.

In embodiments, fluid injector system 104 may include one or more sensing mechanisms, such as one or more sensors, that allow injector management system 102 and/or fluid injector system 104 to detect when a step of a guided workflow has been initiated and/or completed. Injector management system 102 and/or fluid injector system 104 may notify a user of a subsequent step to be completed in a sequence of a guided workflow. The one or more sensors may also be configured to determine if a specific step of a guided workflow has been successfully completed before moving to a subsequent step in the sequence of the guided workflow. If injector management system 102 and/or fluid injector system 104 detects that a step has not been successfully completed or has been skipped, a warning notification may be provided, for example on a user interface, illumination of one or more warning lights (e.g., a red light at or near features associated with a specific step), and/or one or more audible, visual, or tactile warning alerts to the user to indicate that the step has not been correctly completed and any error must but corrected before the guided workflow can progress to a subsequent step. Thus, the guided workflow may allow injector management system 102 and/or fluid injector system 104 to detect a missing step or incorrectly completed step during the sequence of a guided workflow and to revert and guide a user back to a particular step to ensure correct completion of the step for safe use of fluid injector system 104. Where a particular sequence of the steps to be completed is not defined, such as where the steps of a guided workflow may be completed in any sub-sequence or order, injector management system 102 and/or fluid injector system 104 may wait until completion of an entire sub-sequence before sensing and/or providing a notification that one or more steps in the guided workflow have not been completed and/or completed incorrectly.

In embodiments, workstation device 106 may include one or more devices capable of being in communication with injector management system 102, fluid injector system 104, and/or hospital information system 108 via communication network 110. For example, workstation device 106 may include a computing device, such as one or more computers, including a desktop computer, a laptop, tablet, and/or the like. In certain embodiments, at least a portion of the processes executed at workstation device 106 may be executed at a remote server. Workstation device 106 may provide a user interface for controlling operation of fluid injector system 104, including to generate instructions for and/or provide instructions to fluid injector system 104. Workstation device 106 may further display operational parameters of fluid injector system 104 during operation (e.g., during real-time operation) of fluid injector system 104. In various embodiments, workstation device 106 may provide interconnectivity between fluid injector system 104 and other devices or systems, such as a scanner device (see, FIG. 1B). For example, workstation device 106 may include the Certegra® Workstation provided by Bayer. According to various embodiments, workstation device 106 may include one or more display devices, such as a computer monitor, a touchscreen, a heads-up display, a wireless handheld device (e.g., smart pad, smart watch, smart phone, etc.), and/or the like, which may be used to display a user interface (e.g., a graphical user interface (GUI) of a software application), via which a user may interact with workstation device 106 to view parameters and/or control operation of fluid injector system 104.

In embodiments, hospital information system 108 may include one or more devices capable of being in communication with injector management system 102, fluid injector system 104, and/or workstation device 106 via communication network 110. Hospital information system 108 may include one or more subsystems, such as a patient procedure tracking system (e.g., a system that operates a modality worklist, a system that provides patient demographic information for fluid injection procedures and/or medical imaging procedures, etc.), a fluid injection management system, an image archive and communication system (e.g., a picture archive and communication system (PACS)), a radiology information system (RIS), and/or a radiology analytics system (e.g., the Radimetrics® Enterprise Application marketed and sold by Bayer HealthCare LLC). Hospital information system 108 may include one or more databases that stores patient records therein, which may be accessed by devices or systems in environment 100A.

The number and arrangement of systems and/or devices shown in FIG. 1A are provided as an example. There may be additional systems and/or devices, fewer systems and/or devices, different systems and/or devices, or differently arranged systems and/or devices than those shown in FIG. 1A.

Referring now to FIG. 1B, a diagram of a non-limiting embodiment of system 100B for automatically generating or manually inputting an injection protocol for delivery of a medical fluid to a patient during a fluid injection procedure is illustrated. In some embodiments, one or more of the functions described herein with respect to system 100B may be performed (completely or partially) by injector management system 102. In some embodiments, one or more of the functions described with respect to system 100B may be performed by another device or a group of devices separate from and/or including injector management system 102, such as fluid injector system 104, workstation device 106, hospital information system 108, and/or medical imaging system 112. In certain embodiments, the one or more functions may be inputted by a user of system 100B.

As shown in FIG. 1B, system 100B includes injector management system 102, fluid injector system 104, workstation device 106, which includes display device 106A, hospital information system 108, and medical imaging system 112. Injector management system 102 may interconnect and communicate with fluid injector system 104, workstation device 106, hospital information system 108, and/or medical imaging system 112.

As further shown in FIG. 1B, fluid injector system 104 may include one or more display devices, such as display device 104A. Display device 104A may be capable of displaying a user interface (e.g., a web-based user interface, a screen of a user interface, such as a graphical user interface (GUI) screen, etc.), and/or one or more guided workflow steps or processes provided by injector management system 102.

As further shown in FIG. 1B, fluid injector system 104 may include reader device 104B. In embodiments, reader device 104B may include one or more devices that are capable of reading and decoding machine-readable codes (e.g., computer-readable codes, barcodes, such as 2D barcodes, 3D barcodes, quick read (QR) code, etc.). For example, reader device 104B may include a scanner, such as a barcode scanner, an image capture device, such as a camera, a charge-coupled device (CCD) reader, and/or the like.

As further shown in FIG. 1B, workstation device 106 may include display device 106A. In embodiments, display device 106A may be capable of displaying the user interface and have one or more features similar to display device 104A.

In embodiments, medical imaging system 112 may include one or more devices capable of being in communication with injector management system 102, fluid injector system 104, workstation device 106, and/or hospital information system 108 via a communication network. Medical imaging system 112 may include one or more scanners capable of communicating via a communication network and performing medical imaging procedures using a radiological contrast media.

In embodiments, injector management system 102 may include a plurality of applications, (e.g., an informatics application, a data management application, an imaging system connectivity application, etc.) for communicating information with fluid injector system 104, workstation device 106, hospital information system 108, and/or medical imaging system 112. In embodiments, each of the plurality of applications may be associated with an API associated with a respective application that allows fluid injector system 104 to interface with injector management system 102 and/or with hospital information system 108 to provide one or more guided workflow features with regard to operation of fluid injector system 104.

In embodiments, injector management system 102 may provide a user interface that allows a user to control fluid injector system 104. For example, a user may be able to initiate medical imaging, fluid injection, and/or other additional procedures to be performed by fluid injector system 104 via the user interface. The user interface may allow the user to control aspects of the plurality of applications. Injector management system 102 may control fluid injector system 104, such that operations of fluid injector system 104 are carried out automatically based on signals provided by injector management system 102.

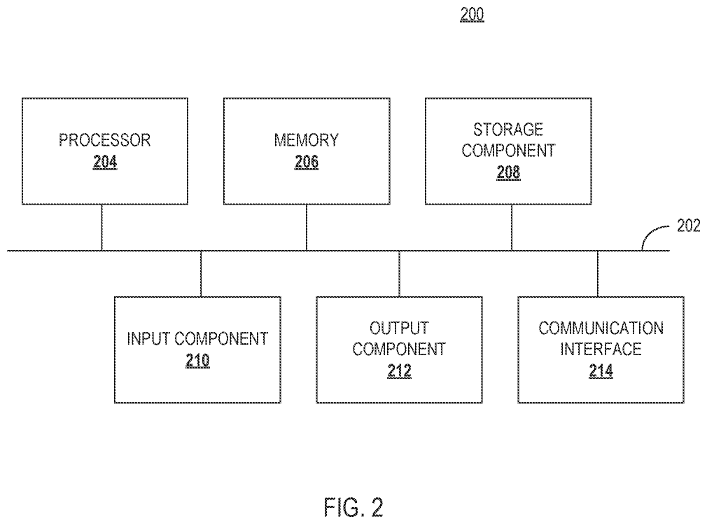

Referring now to FIG. 2, example components of a device 200 are illustrated. Device 200 may correspond to one or more devices of injector management system 102, one or more devices of fluid injector system 104, workstation device 106, hospital information system 108, and/or medical imaging system 112. As shown in FIG. 2, device 200 may include bus 202, processor 204, memory 206, storage component 208, input component 210, output component 212, and communication interface 214.

Bus 202 may include a component that permits communication among the components of device 200. Processor 204 may be implemented in hardware, software (e.g., firmware), or a combination of hardware and software. For example, processor 204 may include a processor, a microprocessor, a digital signal processor (DSP), and/or any processing component that can be programmed to perform a function. Memory 206 may include random access memory (RAM), read only memory (ROM), and/or another type of dynamic or static storage device (e.g., flash memory, magnetic memory, optical memory, etc.) that stores information and/or instructions for use by processor 204. Storage component 208 may store information and/or software related to the operation and use of device 200

Input component 210 may include a component that permits device 200 to receive information, such as via user input. Additionally, or alternatively, input component 210 may include a sensor for sensing information. Output component 212 may include a component that provides output information from device 200 (e.g., a display, a speaker, one or more light-emitting diodes (LEDs), etc.).

Communication interface 214 may include a transceiver-like component that enables device 200 to communicate with (receive information from or provide information to) other devices of the system

Device 200 may perform one or more processes described herein. Device 200 may perform these processes based on processor 204 executing software instructions stored in a computer-readable medium.

Software instructions may be read into memory 206 and/or storage component 208 from another computer-readable medium or from another device via communication interface 214. Additionally, or alternatively, hardwired circuitry may be used in place of or in combination with software instructions to perform one or more processes described herein

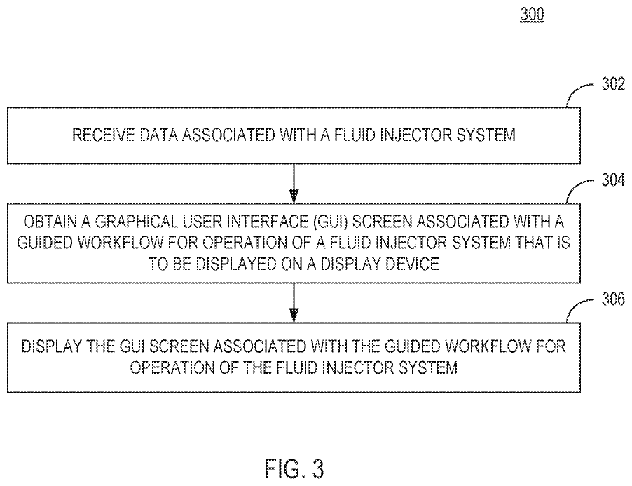

Referring now to FIG. 3, a flowchart of a non-limiting embodiment of a process 300 for providing one or more guided workflow features with regard to a fluid injector system is illustrated. In embodiments, one or more of the steps of process 300 are at least partially performed by injector management system 102, fluid injector system 104, workstation device 106, hospital information system 108, and/or manually by a user of the system.

As shown in FIG. 3, at step 302, process 300 may include receiving data associated with a fluid injector system (e.g., by injector management system 102). In embodiments, injector management system 102 may retrieve data associated with fluid injector system 104 from a data structure of fluid injector system 104.

In certain embodiments, injector management system 102 may receive data associated with one or more components of fluid injector system 104 from a reader device (e.g., reader device 104B). For example, injector management system 102 may receive data associated with one or more components of fluid injector system 104 from a reader device that read (e.g., scanned) a machine-readable code (e.g., a barcode) associated with (e.g., attached to) the one or more components of the fluid injector system. Injector management system 102 may receive the data associated with one or more components of fluid injector system 104 based on the reader device transmitting data associated with one or more components of fluid injector system 104.

In embodiments, the data associated with fluid injector system 104 may include data associated with one or more components of fluid injector system 104 to be used during a fluid injection procedure. For example, the data associated with fluid injector system 104 may include an identifier (e.g., a name, a code, an identification number, a serial number, etc.) of one or more components (e.g., one or more disposable components, such as syringes, fluid path components, tubing, catheter, bulk fluid components, etc.) of fluid injector system 104. Examples and details of such components, including disposable components, may be found in WO2021/173743, WO2021/168076, WO2022/035791, WO2022119837, WO2021/257699, WO2021/257667, and WO2021/050507, the disclosures of which are incorporated by reference herein in their entireties.

As shown in FIG. 3, at step 304, process 300 may include obtaining a GUI screen associated with a guided workflow step or process for operation of the fluid injector system to be displayed on a display device. For example, injector management system 102 may obtain a GUI screen associated with a guided workflow step or process for operation of fluid injector system 104 to be displayed on display device 104A.

In embodiments, a guided workflow may refer to a feature associated with a fluid injector system that guides a user through one or more steps of a procedure, such as a setup (e.g., preparing the fluid injector and/or installing one or more components of the fluid injector system) for a fluid injection protocol, operation of a fluid injection protocol, disposal and/or recycling of various components (syringes or delivery devices, fluid path components, bulk fluid containers, etc.) of a fluid injection procedure after completion of the procedure. In one example, a guided workflow may guide a user through one or more steps associated with a fluid injection procedure. Examples of steps that may benefit or be subject to the guided workflow features according to the present disclosure include, but are not limited to, one or more of a fluid injector system setup process, a patient preparation process, an injection protocol process, a troubleshooting process, a system reconfiguration or teardown process, a process for preparing a fluid injector system for a subsequent fluid injection procedure, a process for recycling one or more components used during a fluid injection procedure, and any combinations thereof.

In embodiments, a guided workflow may include a sequence of GUI screens that provide visual and, in certain embodiments, auditory aids (e.g., instructions, images, text messages, audio instructions, etc.) for conducting a fluid injection protocol with fluid injector system 104. For example, a guided workflow may include one or more features, such as GUI screens, that provide instructions to a user so that the user may efficiently and correctly perform a proper operation of fluid injector system 104. In embodiments, a guided workflow may include a guided workflow for operation of fluid injector system 104, such as a guided workflow for setup of one or more components of fluid injector system 104, for example, programming an injection protocol into the fluid injector system, setting a configuration of one or more disposable components of fluid injector system 104 to be used during a fluid injection procedure, selection of one or more disposable components of fluid injector system 104, setup of one or more fluids to be used during a fluid injection procedure, filling of one or more syringes (or delivery components, such as components of a peristaltic pump system) with one or more fluids to be used during a fluid injection procedure, setup of purging one or more components of fluid injector system 104; a guided workflow for operation of one or more components of fluid injector system 104 or conducting the fluid injection protocol; a guided workflow for disassembly (e.g., teardown, take apart, etc.) of one or more components of fluid injector system 104; a guided workflow for disposal and/or recycling of one or more components of fluid injector system 104, and/or the like. In this way, a guided workflow may guide a user to conduct the various steps in the guided workflow in a manner that is most beneficial for operation of fluid injector system 104, such as the quickest and most efficient way to prepare fluid injector system for a fluid injection procedure which may be based on similarity or differences between a prior fluid injection procedure and a subsequent fluid injection procedure, to minimize time between the fluid injection procedures and maximize a number of fluid injection procedures that may be completed over a time period.

In embodiments, a guided workflow may illustrate steps required to setup fluid injector system 104 to prepare for a patient, i.e. providing functionality to select fluid to be loaded in the syringe, quick access to refill the syringe when fluid injector system 104 determines there is insufficient fluid volume, etc. In embodiments, a guided workflow may illustrate the steps required to conduct a fluid injection protocol. In embodiments, a guided workflow may illustrate the steps required to complete disassembly of one or more components following execution of a fluid injection protocol, e.g., provide guidance to disconnect the patient from the injection apparatus and remove used disposable components from the fluid injector. In embodiments, a guided workflow may illustrate the steps required to prepare used disposable components, such as a syringe, for a recycling process. For example, a guided workflow may illustrate the steps required to remove a plunger and separately recycle a syringe body and/or plunger in an appropriate recycling stream. In other embodiments, the guided workflow may illustrate the steps required to prepare fluid injector system 104 for a subsequent fluid injection procedure, for example select a fluid injection protocol and/or injection parameters, correctly install fluid path components for a selected fluid injection protocol, conduct a fluid fill process, conduct a prime or purge protocol to ensure removal of air from fluid injector system 104, prepare a patient for a fluid injection procedure, etc. Embodiments of the present guided workflow technology may include one or more of the listed workflows or other workflows associated with the fluid injector system.

In embodiments, a guided workflow may provide a way to correct one or more malfunctions of fluid injector system 104. For example, if a system malfunction is experienced by fluid injector system 104, a GUI screen of the guided workflow may provide steps to recover, reconfigure, or otherwise correct fluid injector system 104 from the one or more malfunction. In one example, in a scenario where fluid injector system 104 detects air in one or more components of fluid injector system 104 after a prime or purge sequence and/or during a fluid injection procedure, an air detection guided workflow sequence may provide a sequence of steps to stop an injection procedure and repurge/prime the detected air from the one or more components of fluid injector system 104. The guided workflow may further include one or more workflow steps to determine and/or confirm that the one or more components of fluid injector system 104 has been completely purged of air prior to allowing the fluid injector system to return to a guided workflow associated with a fluid injection protocol.

In particular embodiments, one or more of the guided workflows associated with fluid injector system 104 may be enabled or disabled. For example, in a scenario where a user that is highly experienced with a particular fluid injection procedure of fluid injector system 104, a guided workflow may be disabled and the user may use their familiarity and/or expertise to manipulate fluid injector system 104 through the particular fluid injection procedure. Alternatively, where a user is less experienced or has not performed a particular fluid injection procedure, a guided workflow may be enabled so that fluid injector system 104 may provide a guided workflow to guide the user through the various steps associated with operation of fluid injector system 104.

In embodiments, a GUI screen may display a first message prompting a user to select a user response. For example, a GUI screen may display a first message asking whether or not the user would like to recycle one or more components of fluid injector system 104. Alternatively, if hospital policy favors recycling, the associated guided workflow for a recycling sequence may be enabled to maximize recycling processes In some embodiments, when enabled or upon receiving an input from the user indicating that the user would like to recycle one or more components of fluid injector system 104, the GUI screen may update to display a second message. For example, the GUI screen may display a second message thanking the user for choosing to recycle. In embodiments, the GUI screen may display data associated with a third or subsequent messages incentivizing or guiding the user to recycle one or more components of fluid injector system 104. For example, the GUI screen may display messages such as a discount on future purchases or a certification redeemable by the user, may track amount of medical waste that has been recycled instead of discarded, or may indicate savings to the medical facility in view of the quantity of recycled components. In embodiments, one or more of the GUI screens may display an option to turn recyclability on and/or off.

In some embodiments, one or more of the GUI screens may display data associated with one or more steps for discarding one or more components of fluid injector system 104. For example, a GUI screen may display data including a graphic indicating a container (e.g., recycling bin, biohazard container, sharps container, etc.) where the one or more components of fluid injector system 104 should be discarded, for example based on the material make up of the component. In embodiments, the one or more steps for discarding the one or more components of fluid injector system 104 may be updated based on receiving user input indicating that the user would like to recycle one or more components of fluid injector system 104.

The one or more components of fluid injector system 104 may include a machine-readable code (e.g., a barcode) which may be scanned. In embodiments, in response to scanning the barcode of the one or more components, a GUI screen may be updated to display data specific to the one or more components, for example, how to disassemble and which parts of the one or more component is recyclable and which are to be discarded.

The one or more components of fluid injector system 104 may include different parts made from a plurality of plastic types. In such a situation, one or more of the GUI screens may display data including a plastic type of the part and/or instructions indicating how the part having the plastic type should be handled, i.e., discarded or recycled. In embodiments, a GUI screen may display a graphic indicating a container (e.g., recycling bin, medical waste) where the plastic type should be placed.

In embodiments, fluid injector system 104 may include a software application capable of determining and/or providing a geographic location of where fluid injector system 104 is being operated and the GUI screen may display local, state, country, and/or international standards for recycling a plastic type of the part

In embodiments, various parts of the one or more components of fluid injector system 104 may be contaminated with one or more biohazardous materials, such as residual contrast, and/or patient bodily fluids. In embodiments, one or more of the GUI screens may display data indicating that one or more components of fluid injector system 104 may be contaminated with a biohazardous material and instructions indicating how to discard of the one or more components comprising the biohazardous material. In certain embodiments, a GUI screen may display a graphic indicating a container (e.g., biohazard container, labeled bag) where the biohazardous material should be discarded.

In embodiments, one or more of the GUI screens may display data associated with instructions indicating at least two components of the one or more components of fluid injector system 104 should be discarded in the same container. For example, a GUI screen may display data including instructions and/or a graphic showing the at least two components and a container (e.g., recycling bin, waste basket, biohazard container, sharps container, etc.) where the at least two components should be discarded.

In embodiments, one or more of the GUI screens may display data associated with one or more steps instructing the user to purge and collect remaining medical fluid (e.g., a contrast fluid) from a syringe, dispose or recycle one or more component of the syringe, and/or how to recycle the fluid.

In embodiments, one or more of the guided workflow GUI screens may display one or more steps instructing a user how to disconnect a plunger from the syringe after the fluid is emptied from the syringe, for example, to allow recycling of the syringe body or plunger components and/or to prevent the syringe from being re-used. In embodiments, a GUI screen may display data instructing the user to remove the plunger from the syringe after the fluid is emptied from the syringe, for example by withdrawing the plunger using the fluid injector piston. In embodiments, one or more of the GUI screens may display data associated with one or more steps instructing the user to remove the plunger from the syringe using another syringe, for example by pressurizing a first syringe using a second syringe to pop the plunger out of the first syringe.

In embodiments, one or more of the guided workflow GUI screens may display one or more steps instructing the user to separate a rubber cover from a rigid support ring of a plunger, where the support ring is configured to releasably engage the piston of the fluid injector and the rubber cover provides a seal between the plunger and the syringe sidewall. In some embodiments, one or more of the GUI screens may display one or more steps instructing the user to disconnect the rubber cover from the support ring using a protrusion provided on the back of the head of the injector.

In embodiments, one or more of the GUI screens may display one or more steps instructing the user to recycle the rubber cover and/or the support ring of the plunger, rinse the syringe, and/or recycle the syringe once the plunger is disconnected from the syringe. In embodiments, one or more of the GUI screens may display data associated with instructions indicating how one or more components of fluid injector system 104 may be re-used (e.g., tips and tricks for re-using packaging).

In embodiments, one or more of the GUI screens may display a score, such as a sustainability score (e.g., a green rating) indicating a user's environmental sustainability. For example, in response to completing one or more recycling steps and/or instructions displayed via the GUI screens, a user may input data indicating that the one or more recycling steps have been completed and the score may be determined based on the data input by the user. The score may be based, at least partially, on a number of syringes recycled over a time period and/or an amount (weight) of plastic recycled. In embodiments, one or more of the GUI screens may display a gauge or graph indicating a user's environmental sustainability and or progress towards sustainability goals.

In embodiments, fluid injector system 104 may include instructions for user (IFU) and/or an Operations Manual. The IFU and/or Operations Manual may include a section on recyclability. In embodiments, the IFU and/or Operations Manual may be separate from fluid injector system 104, for example a printed manual. The IFU and/or Operations Manual may include training videos on fluid injector operation procedures and recyclability (e.g., step by step instructions on what to recycle and how to separate components of fluid injector system 104). In embodiments, one or more GUI screens may display the IFU and/or Operations Manual. If the IFU and/or Operations Manual is in the form of a printed manual, they may include a machine-readable code (e.g., a barcode, a quick read (QR) code) that, when scanned, causes a GUI screen to display data associated with the recyclability instructions.

In embodiments, injector management system 102 may determine a guided workflow to display on display device 104A and may obtain appropriate GUI screens for the guided workflow. For example, injector management system 102 may determine an identifier of the guided workflow and may obtain the guided workflow based on determining the identifier.

In embodiments, injector management system 102 may determine a guided workflow to display on display device 104A based on the data associated with fluid injector system 104. For example, injector management system 102 may determine appropriate guided workflow GUI screens to display on display device 104A based on identification of one or more components of fluid injector system 104 to be used during a fluid injection procedure. In some embodiments, injector management system 102 may modify a predetermined sequence of one or more GUI screens of a guided workflow, based on an inputted data associated with the patient and/or fluid injection protocol, to include one or more different GUI screens based on the data associated with fluid injector system 104.

In embodiments, injector management system 102 may receive a user input via a GUI screen of a guided workflow and display another GUI screen associated with the guided workflow based on the user input. For example, injector management system 102 may receive a user input via a graphical element of a first GUI screen of a guided workflow and may display a second GUI screen associated with the guided workflow based on the user input. The second GUI screen may provide information that is sequential to the first GUI screen with regard to the guided workflow for operation of fluid injector system 104. For example, the first GUI screen and the second GUI screen may be part of a sequence of GUI screens for a guided workflow and may be provided in order.

As shown in FIG. 3, at step 306, process 300 may include displaying the GUI screen associated with the guided workflow for operation of the fluid injector system. In some embodiments, injector management system 102 may display one or more GUI screens associated with a guided workflow for operation of fluid injector system 104 on one or more display devices. For example, injector management system 102 may display one or more GUI screens associated with a guided workflow for operation of fluid injector system 104 on display device 104A. In one example, injector management system 102 may display a first GUI screen associated with a guided workflow on display device 104A and injector management system 102 may receive a user input via the first GUI screen. Subsequently, injector management system 102 may obtain a second GUI screen associated with the guided workflow and display the second GUI screen on display device 104A in response to the user input and programmed fluid injection protocol.

In embodiments, injector management system 102 may display at least a portion of a GUI screen associated with a guided workflow on a first display device. Injector management system 102 may display at least a first portion of a GUI screen on a first display device and injector management system 102 may display at least a second portion of the GUI screen on a second display device. In embodiments, the first display device may be a display device of a system or device that is different than the system or device that includes the second display device. For example, the first display device may be a display device associated with the injector and the second display device may be a display device in an imaging suite.

In embodiments, injector management system 102 may display at least a first portion of a GUI screen on a first display device and/or injector management system 102 may display at least a second portion of the GUI screen on a second display device based on a user input, for example, by receiving the user input via a graphical element of a GUI screen, such as from part of a guided workflow as described herein. In embodiments, injector management system 102 may display at least the first portion of the first GUI screen on a first display device of fluid injector system 104 and/or injector management system 102 may display at least the second portion of the first GUI screen on a second display device (e.g., of a system, such as hospital information system 108, of a device, such as workstation device 106, etc.) that is independent of (e.g., not part of) fluid injector system 104. In embodiments, injector management system 102 may display at least a first portion of a second GUI screen on a first display device of fluid injector system based on receiving a user input via the graphical element of a first GUI screen or an input device (e.g., a button, a touchscreen, a control, etc.) and/or display at least a second portion of the second GUI screen on a second display device (e.g., of a system, such as hospital information system 108, of a device, such as workstation device 106, etc.) that is independent of fluid injector system based on receiving a user input via a graphical element of the first GUI screen or the input device.

In embodiments, injector management system 102 may execute an operation of fluid injector system 104, for example based on a user input received via a GUI screen as part of a guided workflow process, for example via a graphical element. The injector management system 102 may generate a control signal based on a user input received via a GUI screen and may execute the operation of fluid injector system 104 by transmitting control signal to fluid injector system 104.

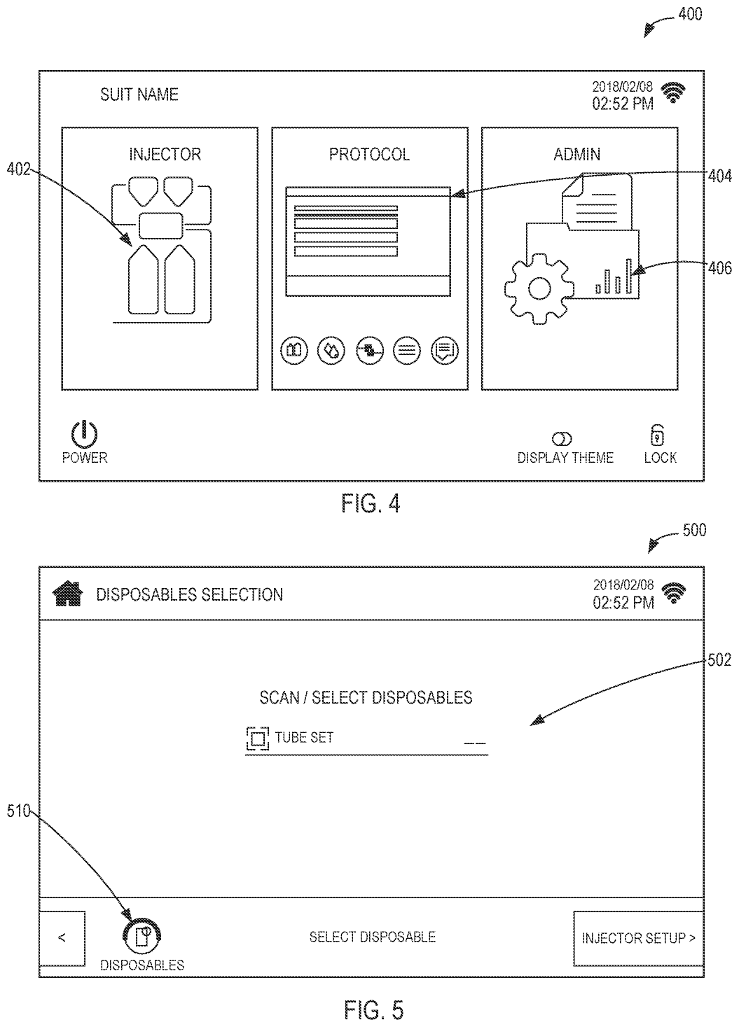

Referring now to FIGS. 4-17D, FIGS. 4-17D are illustrations of non-limiting embodiments of GUI screens provided as a part of one or more guided workflow features with regard to fluid injector system 104 as described herein. The precise style, arrangement, and configuration of the GUI screens and elements are exemplary and do not prescribe a sequence of operation, unless specified otherwise. In embodiments, one or more elements of one GUI screen described herein may be combined with that of one or more other GUI screens. A guided workflow as described herein may include one or more of the GUI screens described in FIGS. 4-17D.

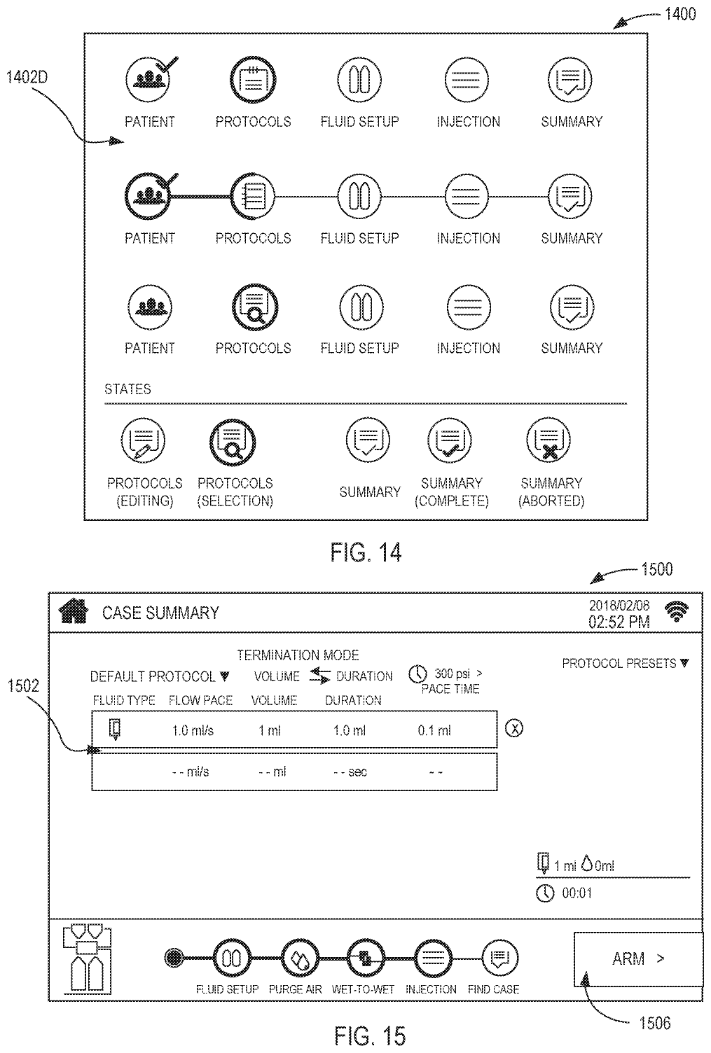

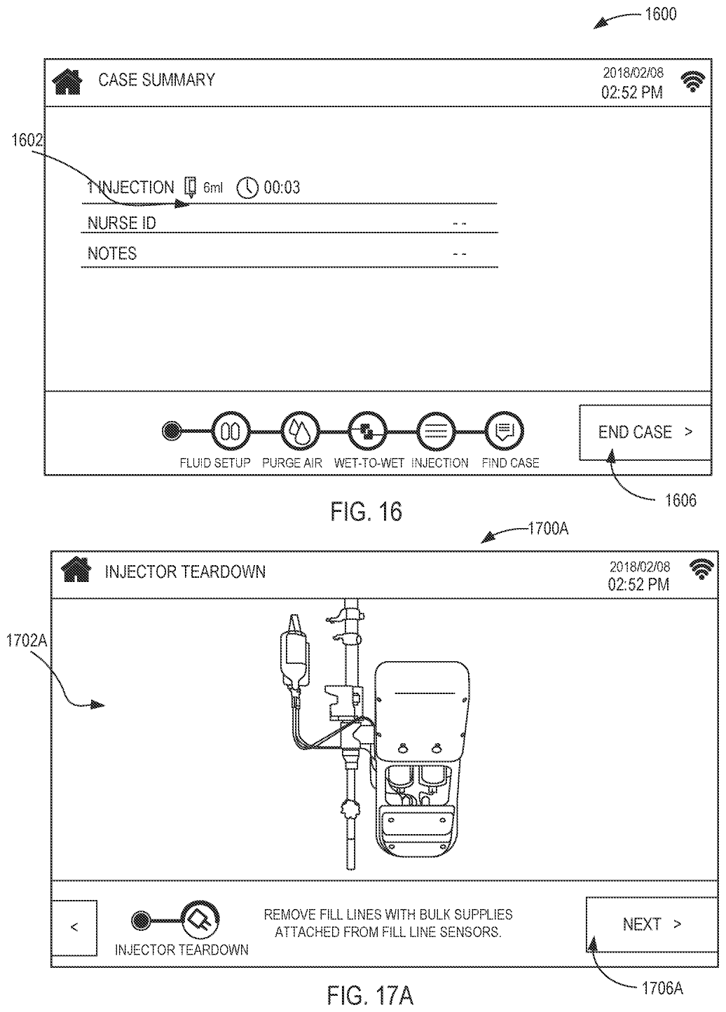

Referring now to FIG. 4, FIG. 4 is an illustration of a non-limiting embodiment of an introductory GUI screen of a guided workflow for operation of fluid injector system 104 as described herein. As shown in FIG. 4, GUI screen 400 may include one or more graphical elements 402, 404, and 406. In embodiments, graphical element 402 may allow a user to initiate a guided workflow sequence for setup, operation, or disassembly of one or more components of fluid injector system 104. For example, upon receiving a selection of graphical element 402, injector management system 102 may provide a guided workflow for operation of fluid injector system 104. Graphical element 404 may allow a user to initiate a guided workflow for operation of fluid injector system 104 including a sequence of steps of a fluid injection protocol (e.g., GUI screen 1400 shown in FIG. 14, GUI screen 1500 shown in FIG. 15, or GUI screen 1600 shown in FIG. 16). In embodiments, graphical element 406 may allow a user to initiate a guided workflow for operation of fluid injector system 104 including a sequence of steps associated with a configuration of one or more components of fluid injector system 104.

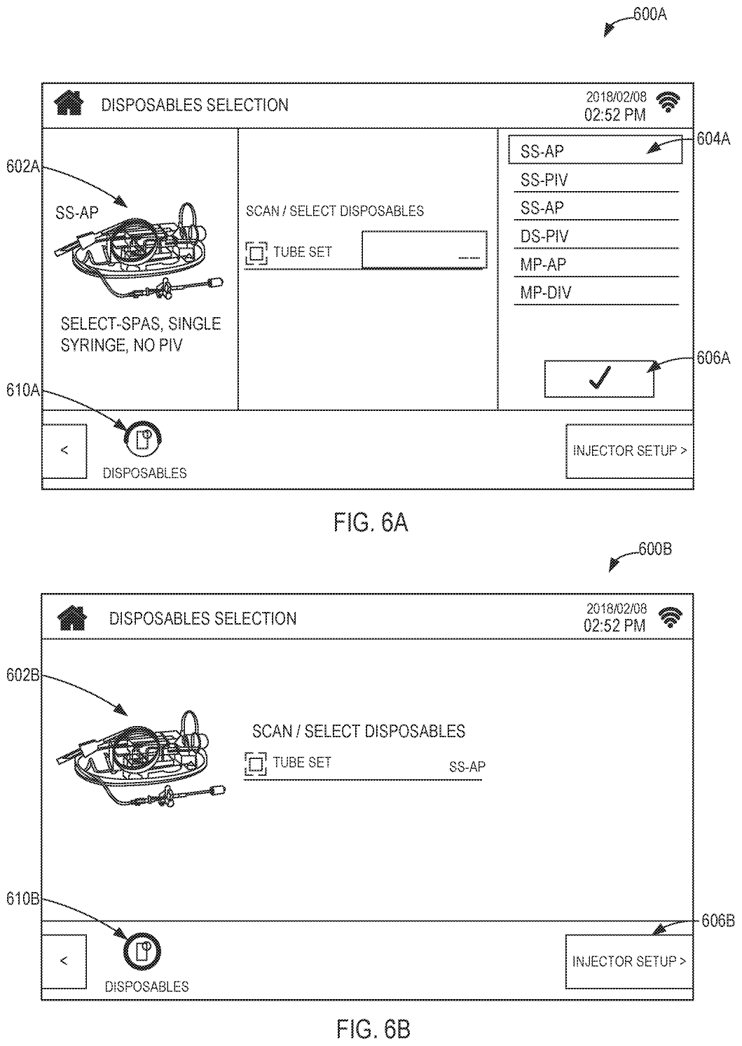

Referring now to FIGS. 5-6B, an embodiment of a guided workflow for setting a configuration of one or more disposable components of fluid injector system 104 is illustrated. As used herein, “disposable components” for a fluid injector system include, those fluid injector components that are used once or over a set number of fluid injections or over a set time and are then discarded and replaced with new, sterile disposable fluid injector components for a subsequent injection procedure or series of injection procedures. Examples of disposable components include, but are not limited to, syringes, peristaltic pump components, tubing set components (tubing, stopcocks, connector assemblies, spikes, air bubble detection/delay components, tubing cassettes, dust caps, prime tubes, PIV valves), pressure jackets, and the like. As shown in FIG. 5, GUI screen 500 may include graphical elements 502 and 510. Graphical element 502 may display a prompt for a user to select a graphical identifier of and/or scan a barcode of one or more components of fluid injector system 104, such as a syringe type, tubing type, medical fluid type, etc. Additionally or alternatively, graphical element 502 may display one or more text messages (e.g., “select disposable”) regarding identification and/or setup of the one or more disposable components. In embodiments, graphical element 510 may display a status of progress of the guided workflow for setting the configuration of the one or more disposable components.

With reference to FIG. 5, upon receiving user input regarding selection of and/or receiving a barcode identification of a disposable component of fluid injector system 104, injector management system 102 may then display a GUI screen that is next in a sequence of GUI screens for a guided workflow for setting a configuration of one or more disposable components. For example, injector management system 102 may provide GUI screen 600A (FIG. 6A) based on receiving the user input and/or the barcode identification of the disposable component.

Referring now to FIG. 6A, a GUI screen 600A corresponding to an embodiment of a screen for selection of one or more disposable components of fluid injector system 104 is illustrated. GUI screen 600A may include one or more graphical elements, such as graphical elements 602A, 606A, and 610A. In embodiments, graphical element 602A may display a selection of one or more disposable components of fluid injector system 104, for example based on receiving the user input and/or barcode reading of the disposable component (see, FIG. 5). In embodiments, graphical element 602A may display an image of a selected disposable component. Additionally or alternatively, graphical element 602A may display a text message regarding the selected disposable component. Graphical element 604A may display the possible disposable elements that could have been selected in the step associated with the guided workflow step illustrated in FIG. 5, with the selected disposable element highlighted. Graphical element 604A may allow a user to change the selected element, should it be determined that the originally selected disposable element is incorrect. Graphical element 606A may display a prompt for a user to confirm the selection of the disposable component based on the user input and/or barcode reading of the disposable component, for example by clicking on a checkmark illustrated in 606A, to continue to the next guided workflow GUI screen. Graphical element 610A may display an updated status of progress regarding setting the configuration of the one or more disposable components of fluid injector system 104.

Upon confirming a selection of an identifier of and/or barcode reading of a disposable component, injector management system 102 may provide a GUI screen that is next in a sequence of GUI screens moving to a guided workflow for setting a configuration of one or more additional disposable components of fluid injector system 104. For example, injector management system 102 may provide GUI screen 600B for user input and/or a barcode reading of one or more additional disposable component of fluid injector system 104. A user may return to the GUI guided workflow screen for selecting additional disposable components to be utilized in a fluid injection procedure, for example by returning to the GUI screen shown in FIG. 5. Alternatively, if all disposable components for a particular fluid injection procedure, as determined by a user or injector management system 102, are selected, the guided workflow may move to a confirmation screen, as shown in FIG. 6B.

Referring now to FIG. 6B, a disposable component confirmation GUI screen 600B which may be a subsequent GUI screen for listing all of the one or more disposable components selected for fluid injector system 104 is illustrated. GUI screen 600B may include graphical elements 602B, 606B, and 610B. Graphical element 602B may display one or more identifiers of all previously selected disposable component of fluid injector system 104 based on receiving the user input/barcode readings, as described herein, associated with the various disposable components for a fluid injection protocol.

In embodiments, graphical element 606B may allow a user to select to proceed to a guided workflow for setup of the various selected disposable components of fluid injector system 104. Graphical element 610B may display an updated status of progress for the guided workflow regarding selecting the configuration of the one or more disposable components of fluid injector system 104 (e.g., 100% complete).

In embodiments, upon receiving confirmation that all disposable injector components have been selected for a fluid injection protocol for fluid injector system 104, injector management system 102 may provide the next GUI screen of a sequence of GUI screens for a guided workflow for setup of one or more fluids and disposable injector components to be used during a fluid injection procedure of fluid injector system 104. For example, injector management system 102 may provide GUI screen 700A based on receiving confirmation via graphical element 606B to continue to a guided workflow for setup of one or more fluids and setup of selected disposable components to be used during a fluid injection procedure.

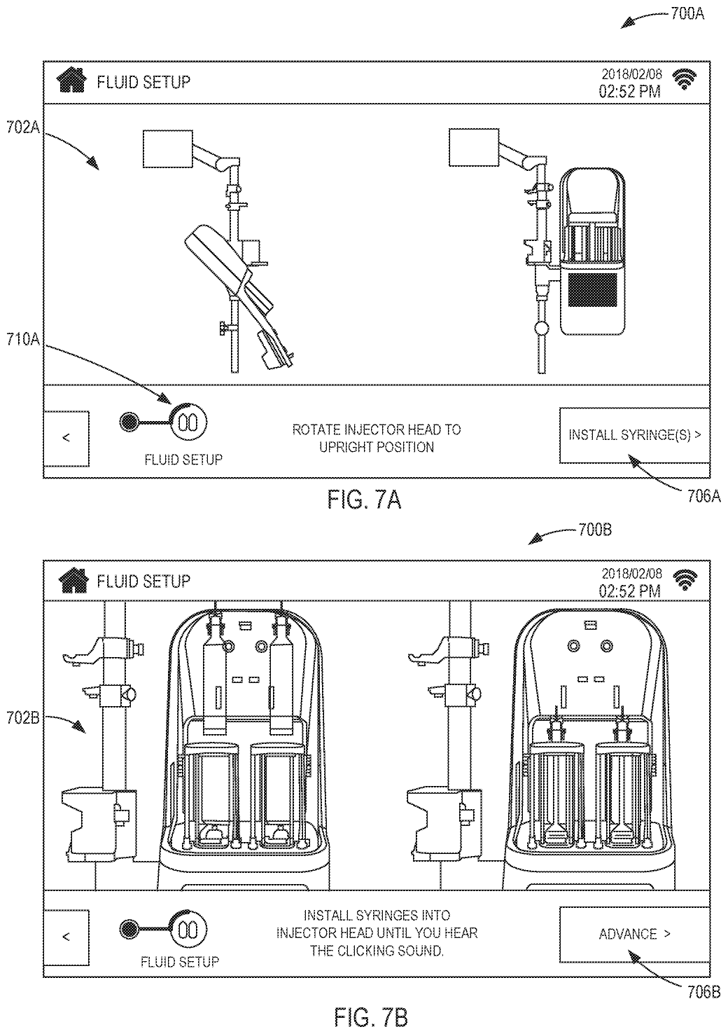

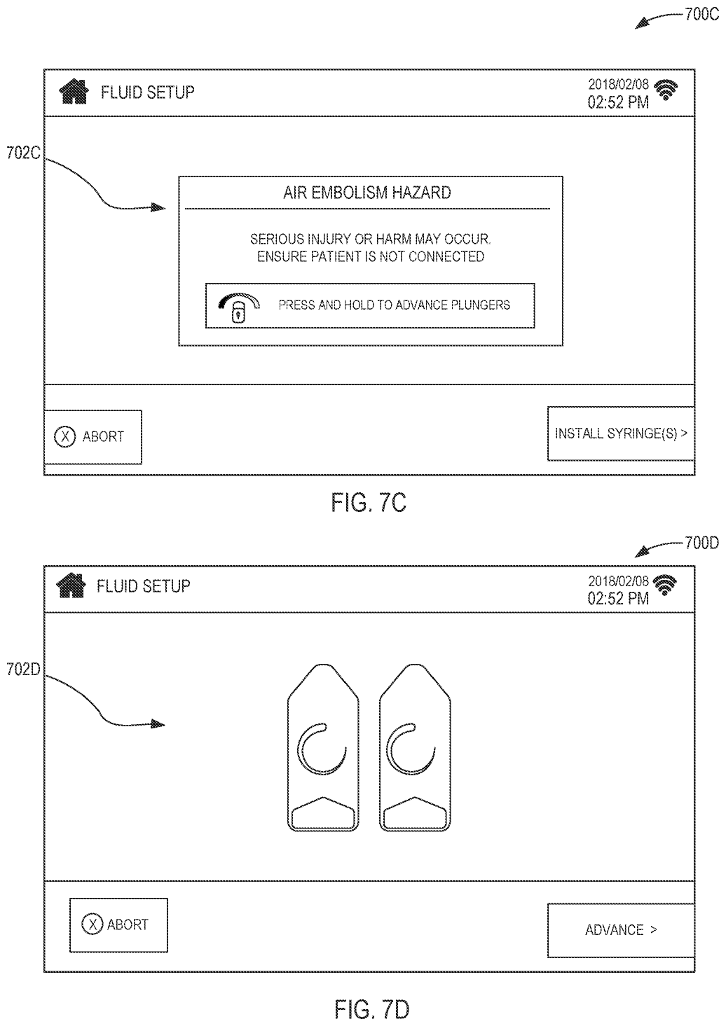

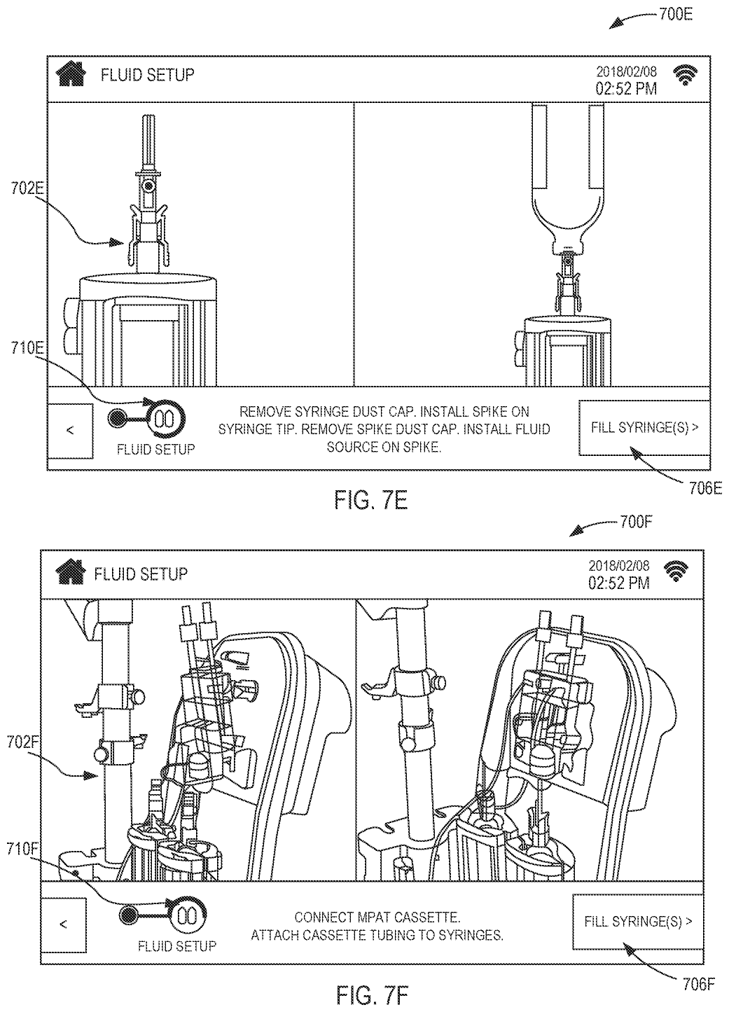

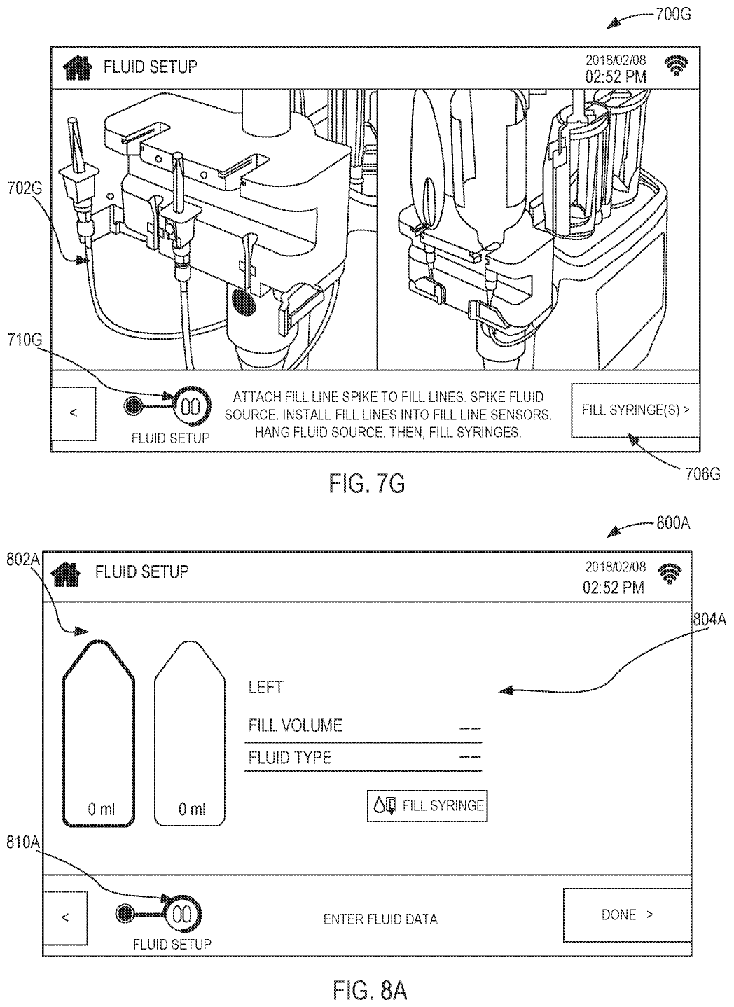

Referring now to FIG. 7A-7G, GUI screens 700A-700G illustrate an embodiment of a guided workflow for setup of one or more fluids and disposable components selected to be used during a fluid injection procedure of fluid injector system 104 are illustrated. In embodiments, the guided workflow shown in FIGS. 7A-7G may be displayed after the completion of the guided workflow shown in FIG. 6A-6B. That is, injector management system 102 may progress to a guided workflow for setup of one or more fluids and setup of the disposable components to be used during a fluid injection procedure.

Referring now to FIGS. 7A-7B, GUI screen 700A may be a first GUI screen for a guided workflow for setup of one or more fluids/disposable components to be used during a fluid injection procedure is illustrated. As shown in FIGS. 7A-7B, GUI screens 700A and 700B may include graphical elements 702A,B, 706A,B, and 710A associated with installation of one or more syringes onto the fluid injector. In embodiments, graphical element 702A may display an image of a configuration of fluid injector system 104. Additionally or alternatively, graphical element 702A may display a text message to guide a user through a setup of the one or more syringes of fluid injector system 104.

Graphical element 706A may allow a user to select to proceed to a subsequent GUI screen of a guided workflow for setup of one or more syringes to be used during a fluid injection procedure. For example, upon receiving a selection of graphical element 706A, injector management system 102 may provide GUI screen 700B of the guided workflow. In some embodiments, graphical element 710A may display a status of progress of the guided workflow for setup of one or more fluids and disposable components to be used during a fluid injection.

Referring now to FIG. 7B, GUI screen 700B continuing a guided workflow for setup of one or more fluids and disposable components is illustrated. GUI screen 700B may include at least graphical elements 702B and 706B. For example, graphical element 702B may display an image of proper installation of one or more syringes into pressure jackets and syringe ports associated with fluid injector system 104. Additionally or alternatively, graphical element 702B may display a text message (e.g., “install syringes into injector head until you hear the clicking sound”) regarding proper installation of the one or more syringes.

Graphical element 706B may allow a user to proceed to a subsequent GUI screen of a guided workflow after successful completion of the installation of the syringes. In certain embodiments where the syringes have not been properly installed, feature 706B may not be selectable (e.g., may appear “greyed out” as in 706A and not respond to selection by the user) and the user may be prompted to correctly complete the workflow step before feature 706B may be selected Upon successful completion of the step associated with the guided workflow illustrated in FIGS. 7A and 7B, selection of graphical element 706B may prompt injector management system 102 to proceed to GUI screen 700C of the guided workflow for setup of one or more fluids/disposable components for the fluid injection procedure.

Referring now to FIG. 7C, GUI screen 700C for a guided workflow for setup of one or more fluids/disposable components is illustrated to be used during a fluid injection procedure. GUI screen 700C may include graphical element 702C for example a warning when the installed one or more syringes are not completely filled with a medical fluid (e.g. an air embolism hazard warning). Additional text (e.g., “serious injury or harm may occur, ensure patient is not connected”) regarding the warning associated with the determination that air may be in one or more of the one or more syringes may be displayed to prompt a user to fill or prime the one or more syringes. Graphical element 702C may allow a user to select to proceed to a subsequent GUI screen of a guided workflow to fill the one or more syringes with one or more fluids to be used during a fluid injection procedure. For example, upon selection of graphical element 702C, injector management system 102 may proceed to GUI screen 700D of a guided workflow for filling the one or more syringes with the one or more fluids.

Referring now to FIG. 7D, GUI screen 700D for the guided workflow for setup of one or more fluids is illustrated. Graphical elements 702D may display an image of one or more syringes of fluid injector system 104 to be filled with a fluid as a waiting screen (e.g., loading screen). A user may select to proceed with the guided workflow or abort the fluid setup of the fluid injection procedure by selecting a graphical element associated with the option.

Referring now to FIG. 7E, GUI screen 700E for the guided workflow for setup of one or more fluids is illustrated. GUI screen 700E may include graphical elements 702E, 706E, and 710E illustrating installation of a spike on a syringe and engaging and filling the syringe with a fluid from a bulk fluid source. Graphical element 702E may display an image installation of a filling spike onto a distal connector component of a syringe and installation of a bulk fluid source on the spike of the syringe. Additionally or alternatively, graphical element 702E may display a text message guiding a user through the process illustrated in 702E, for example, “remove syringe dust cap, install spike on syringe tip, remove spike dust cap, install fluid source on spike”, etc.

Graphical element 706E may allow a user to proceed to a subsequent GUI screen of a guided workflow for setup of one or more fluids once the process illustrated in FIG. 7E has been successfully completed, for example as determined by the user or injector management system 102. For example, upon selection of graphical element 706E, injector management system 102 may provide GUI screen 700F of a guided workflow for setup of one or more fluids to be used during a fluid injection procedure. Graphical element 710E may display a status of progress of the guided workflow for setup of one or more fluids.

Referring now to FIG. 7F, GUI screen 700F guided workflow for setup of one or more disposable components to be used during a fluid injection procedure after filling of the one or more syringes with a medical fluid is illustrated. GUI screen 700F may include graphical elements 702F, 706F, and 710F. Graphical element 702F may display an image for installation of one or more components (e.g., a multi-patient disposable set (MPAT) cassette) of fluid injector system 104 including images of the process for properly engaging the one or more component and/or the properly engaged one or more component. Graphical element 702F may display a text message (e.g., “connect MPAT cassette, attach cassette tubing to syringes”) regarding the workflow for the setup of the one or more components.

Graphical element 706F may allow a user to proceed to a subsequent GUI screen of a guided workflow for setup of one or more fluids/components upon successful completion of the setup process associated with the guided workflow associated with the current GUI screen. For example, upon receiving a selection of graphical element 706F, injector management system 102 may proceed to a subsequent guided workflow step, for example as shown in GUI screen 700G, for further setup of one or more fluids and/or components to be used during a fluid injection procedure. As described herein, graphical element 710F may display an updated, real time status of progress of the guided workflow for setup of one or more fluids and/or components to be used during a fluid injection procedure of fluid injector system 104.

Referring now to FIG. 7G, GUI screen 700G for a guided workflow for setup of one or more fluids and/or components to be used during a fluid injection procedure is illustrated. GUI screen 700G may include graphical elements 702G, 706G, and 710G. Graphical element 702G may display an image of a setup procedure of one or more components, for example, one or more spikes and associated fill lines, bulk fluid container holder, and syringes of fluid injector system 104 to allow spiking of the bulk fluid containers and filling of the one or more syringes with medical fluids, as described herein. Graphical element 702G may display a text message (e.g., “attach fill line spike to fill lines, spike fluid source, install fill lines into fill line sensors, hang fluid source, then, fill syringes” in FIG. 7G) guiding a user through the workflow associated with filling fluid into the syringes via one or more components of fluid injector system 104.

In embodiments, graphical element 706G may allow a user to proceed to a subsequent GUI screen of a guided workflow for setup of one or more fluids to be used during a fluid injection procedure of fluid injector system 104. In embodiments, graphical element 710G may display an updated, real time status of progress of the guided workflow for setup of one or more fluids and/or components.

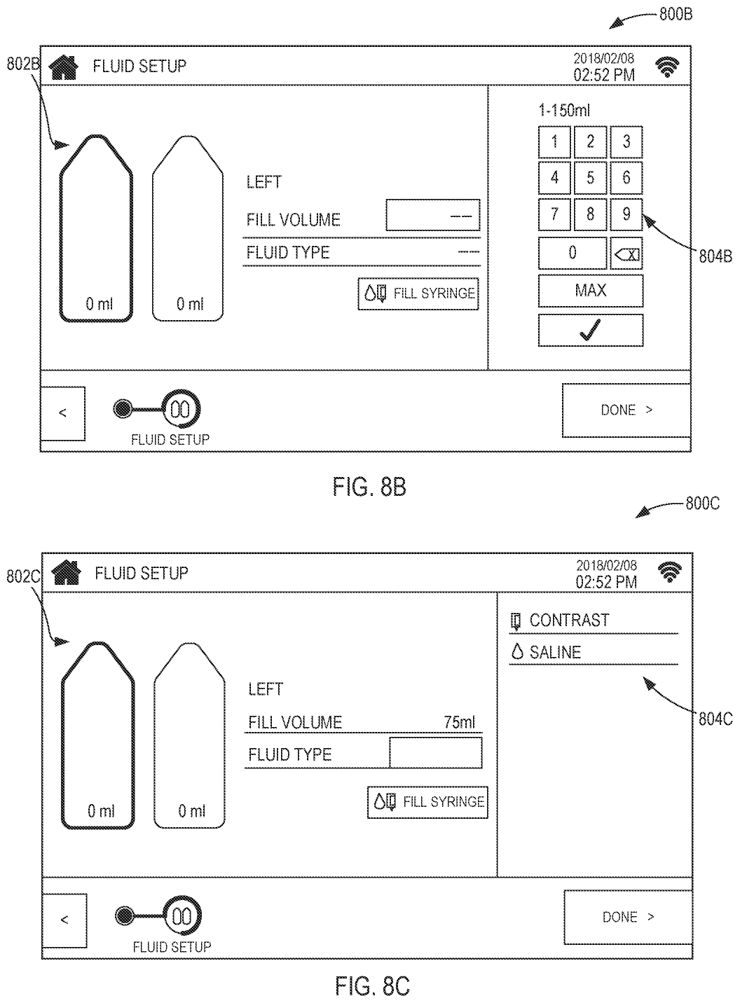

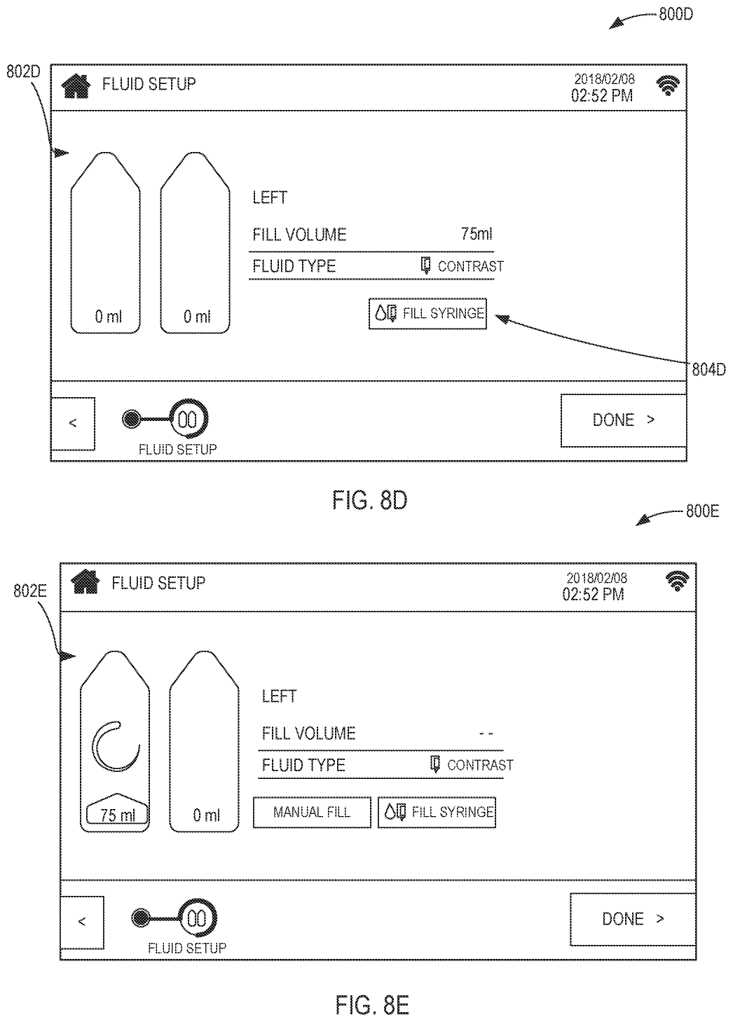

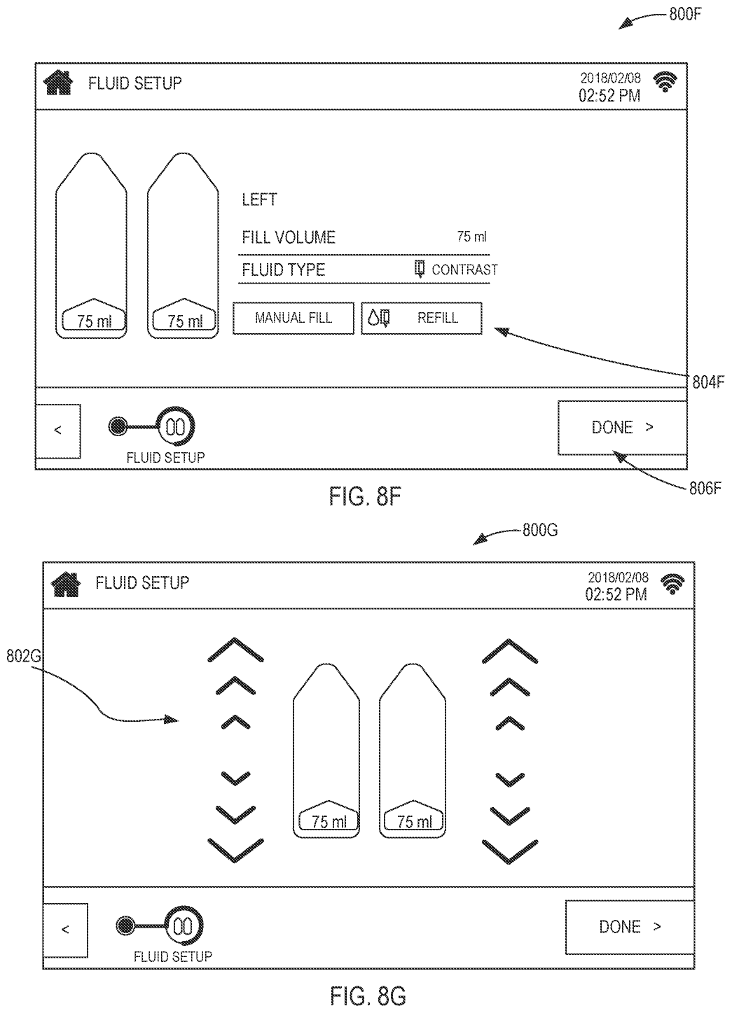

Referring now to FIGS. 8A-8G, GUI screens 800A-800G of a guided workflow are illustrated for filling of one or more syringes with one or more fluids to be used during a fluid injection procedure. In some embodiments, the guided workflow shown in FIGS. 8A-8G may follow the guided workflow show in FIG. 7A-7G. For example, upon completion of the guided workflow of previous GUI screen(s), injector management system 102 may progress to a guided workflow for filling of one or more syringes with one or more fluids to be used during a fluid injection procedure.

Referring now to FIG. 8A, GUI screen 800A may be an initial GUI screen for a guided workflow for filling of one or more syringes with one or more fluids to be used during a fluid injection procedure. GUI screen 800A may include graphical elements 802A, 804A, and 810A. Graphical element 802A may display an image of one or more components of fluid injector system 104 (e.g. left syringe, right syringe or other fluid delivery device, such as a peristaltic pump component). Graphical element 802A may display a text message (e.g., “enter fluid data”) regarding the workflow for filling the one or more syringes. Graphical element 802A may allow selection of a left syringe for filling with a first medical fluid and/or selection of the right syringe for filing of a second medical fluid according to the fluid injection protocol. In embodiments, graphical element 804A may display a prompt for a user to select or enter one or more properties of a component of fluid injector system 104, for example, fill volume or fluid type for the left syringe or right syringe. In certain embodiments, graphical element 804A may provide a fill volume or a fluid type for filling a syringe from one or more of a previously programmed fluid injection protocol and/or a barcode read from a fluid container. Graphical element 810A may display a status of progress of the guided workflow as described herein.

Referring now to FIG. 8B, GUI screen 800B, for a guided workflow for filling of one or more syringes with one or more fluids is illustrated. Graphical element 802B may highlight the syringe to be filled and provide for entry of a fill volume for filling the syringe and the fluid type. Graphical element 804B may display a user input field for entering fill volume for the highlighted syringe or, alternatively, the fill volume may be entered by injector management system 102 based on a previously programmed or entered injection protocol which may be selected by a user using a graphical element (not shown).

Referring now to FIG. 8C, GUI screen 800C, for a guided workflow for filling of one or more syringes with one or more fluids is illustrated. In embodiments, graphical element 802C may provide for entry of fluid type for filling a syringe. In embodiments, graphical element 804C may display a selection of fluid type for filling the selected syringe.

Referring now to FIG. 8D, GUI screen 800D for a guided workflow for initiating filling of one or more syringes with one or more fluids is illustrated. Graphical element 802D may display an image of one or more components as described herein (e.g. left syringe, right syringe). Graphical element 804D may display a prompt for a user to fill syringe and proceed to a subsequent GUI screen of the guided workflow. For example, upon receiving a selection of graphical element 804D, injector management system 102 may initiate filling of the indicated syringe with the chosen volume of medical fluid as shown on GUI screen 800E.

Referring now to FIG. 8E, GUI screen 800E for a guided workflow for filling of one or more syringes with one or more fluids to be used during a fluid injection procedure is shown. Graphical element 802E may display a selected fill volume of medical fluid for one or both of the syringes of fluid injector system 104. Upon completion of programming or entering the fill volume of selected medical fluid for the one or more syringes (e.g., 75 ml), injector management system 102 may provide GUI screen 800F of a guided workflow for setup of one or more fluids to be used during a fluid injection procedure.

Referring now to FIG. 8F, GUI screen 800F, for a guided workflow for filling of one or more syringes with one or more fluids to be used during a fluid injection procedure is illustrated. GUI screen 800F may include graphical elements 802F, 804F, and 810F. Graphical element 802F may provide a programmed or selected volume of medical fluid to be filled into the one or more syringes (e.g. 75 ml, 75 ml). In certain embodiments, a user may select to manually fill the one or more syringes by selecting the manual fill feature. The user may then be guided to a GUI screen allowing manual filling of one or both syringes with the associated fluid. Alternatively in a multi-patient setup, a user may select to refill one or both syringes if the volume of fluid in the syringe is too low to complete the currently programmed procedure, for example, due to injection of a volume of fluid in a previous fluid injection procedure or to automatically refill the one or more syringes to the selected volume at the end of a fluid injection procedure. Graphical element 806F may allow a user to select to proceed to a subsequent GUI screen of a guided workflow for setup of one or more fluids to be used during a fluid injection procedure of fluid injector system 104.

Referring now to FIG. 8G, GUI screen 800G illustrates a step of a guided workflow for filling of one or more syringes with one or more fluids to be used during a fluid injection procedure of fluid injector system 104. Graphical element 802G may display a configuration of one or more components, such as a left syringe and right syringe of fluid injector system 104. Graphical element 802G may indicate the status of a procedure for filling one or more syringes, for example by illustrating that fluid injector system 104 is retracting the piston associated with each syringe to fill the syringe with the selected volume of the selected fluid as indicated by the arrows next to the syringe.

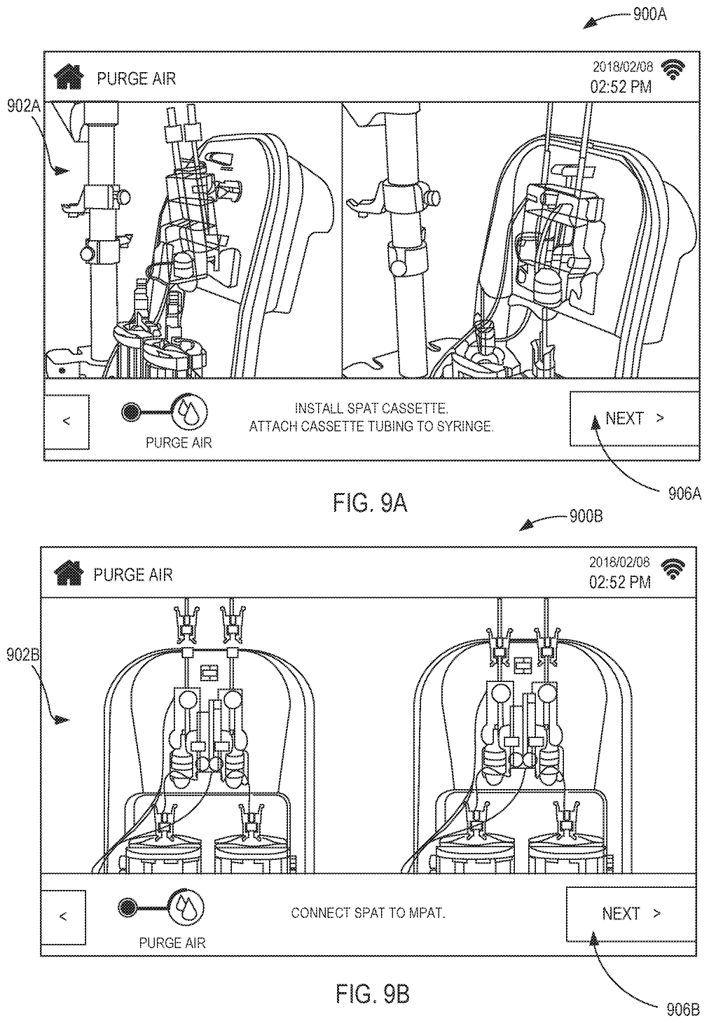

Referring now to FIGS. 9A-9B, illustrations of non-limiting embodiments of GUI screens 900A-900B of a guided workflow for connecting one or more tubing set components of a fluid injection system are shown. Upon completion of the guided workflow for filling of one or more syringes with one or more fluids to be used during a fluid injection procedure, injector management system 102 may progress to a guided workflow for purging the components of the fluid injection system.

Referring now to FIG. 9A, GUI screen 900A for a guided workflow for purging of one or more components of fluid injector system 104 is illustrated. FIG. 9A illustrates guided workflow for installation of a fluid path component in the form of a cassette for a single patient (SPAT) fluid injection procedure. The process illustrated in FIG. 9A is analogous to that illustrated in FIG. 7F for installation of a fluid path component in the form of a cassette for a multi-patient (MPAT) injection procedure. In the guided workflow illustrated in FIG. 9A, the syringes have been filled with associated medical fluids (see workflow associated with FIGS. 8A-8G) and are ready for purging after installation of all the fluid path components (e.g., fluid path component cassette shown) on a receiving surface of an injector head of fluid injector system 104. Graphical element 902B may display an image associated with attaching a single patient tubing set to another component installed on fluid injector system 104 and/or graphical element 902A may display instructional text message regarding the guided workflow step. Graphical element 906A may allow a user to proceed to the next step associated with the guided workflow for purging air from fluid injector system 104.

Referring now to FIG. 9B, GUI screen 900B for a guided workflow for installation of a single patient fluid line (SPAT) to a multi-patient tubing set cassette is illustrated for fluid injector system 104. The workflow associate with FIG. 9B would be subsequent to installation of the multi-patient cassette component illustrated in the GUI screen illustrated in FIG. 7F. Graphical element 902B may display an image associated with attaching a single patient tubing set to the multi-patient cassette component installed on fluid injector system 104. Graphical element 902B may display instructional text message (e.g., “Connect SPAT to MPAT” in FIG. 9B) regarding the guided workflow step. In embodiments, graphical element 906B may allow a user to select to proceed to a subsequent guided workflow step upon proper completion of the workflow step illustrated in FIG. 9B. Upon receiving a selection via graphical element 906B, injector management system 102 may provide a subsequent GUI screen for a guided workflow for purging one or more components of fluid injector system 104.

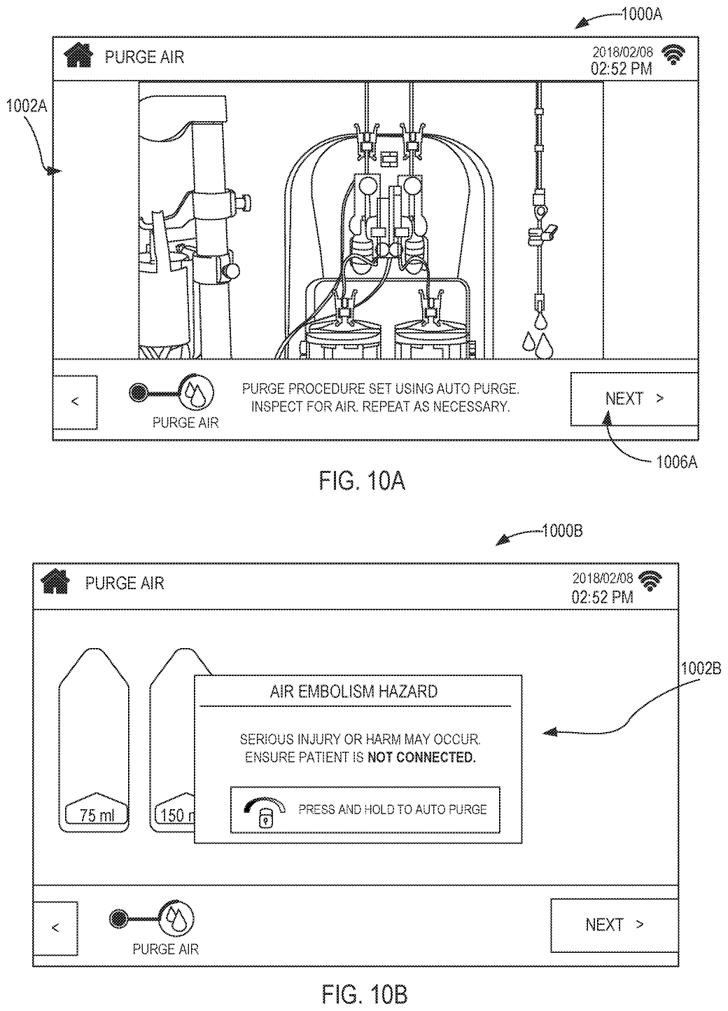

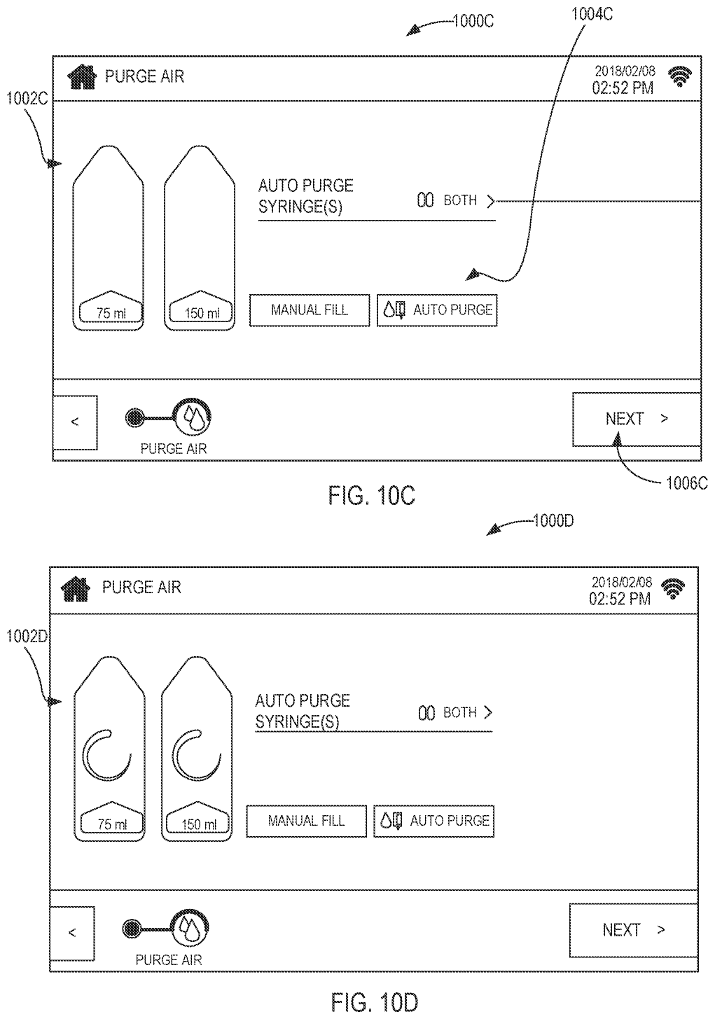

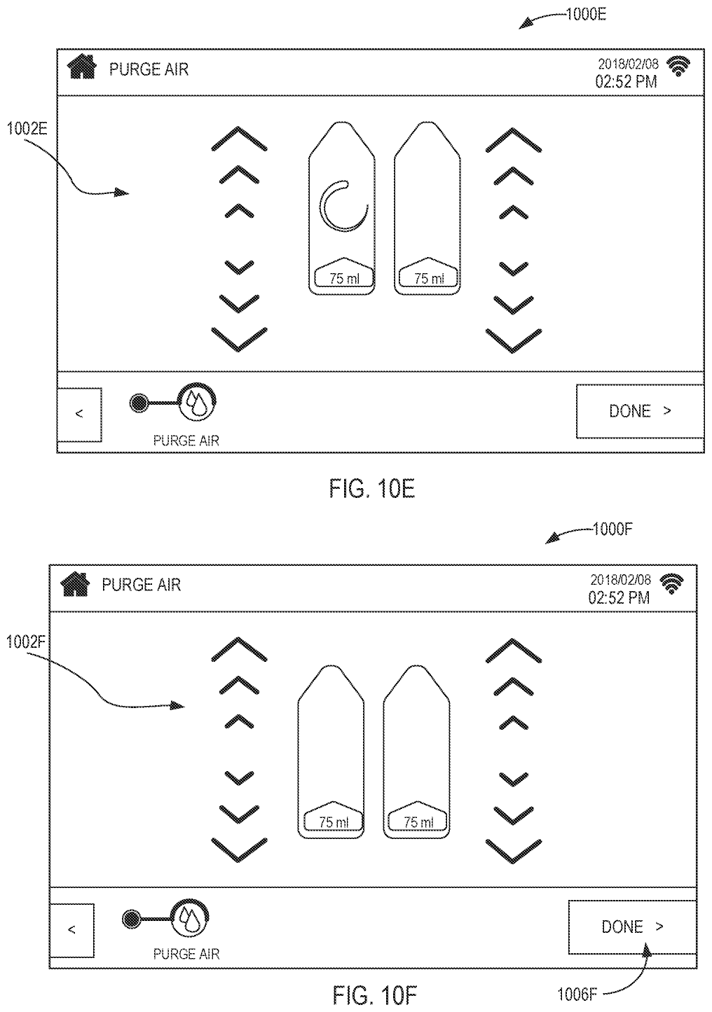

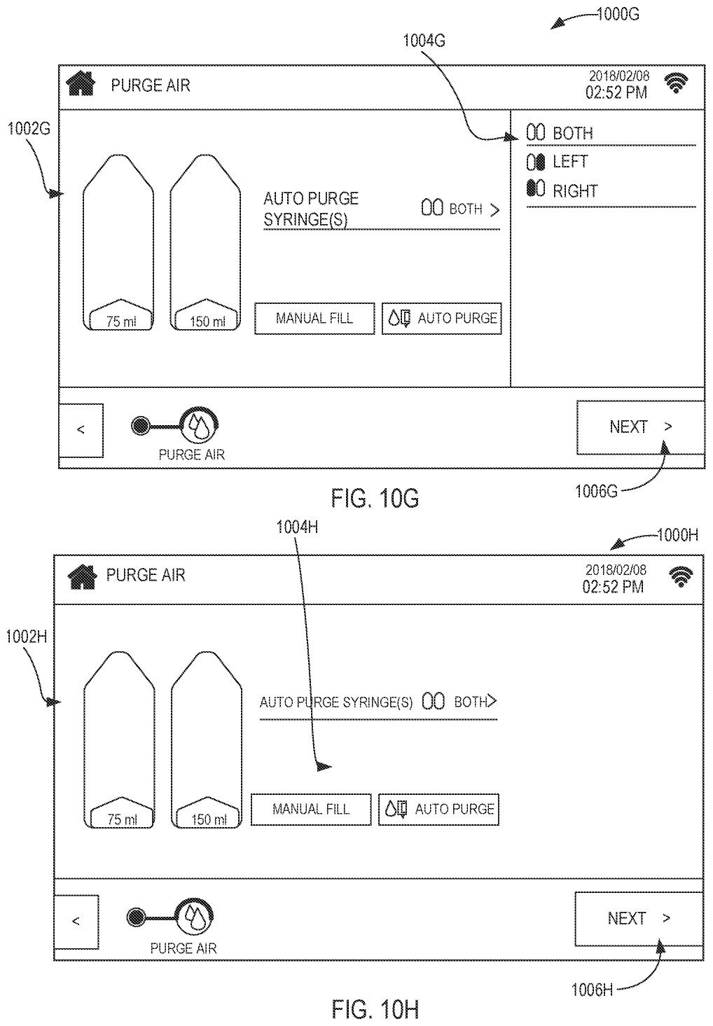

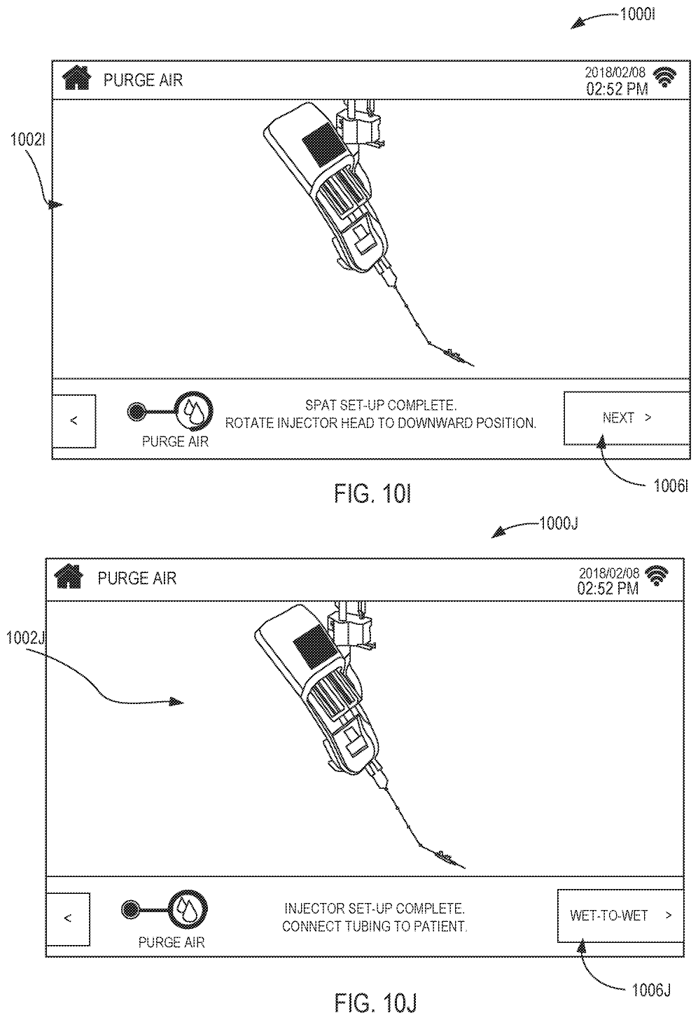

Referring now to FIGS. 10A-10J, GUI screens of a guided workflow for allowing fluid injector system 104 to manually purge or automatically purge air from one or more components of fluid injector system 104 are illustrated. In embodiments, the guided workflow process shown in FIGS. 10A-10J may follow the guided workflow in FIGS. 9A-9B.

Referring now to FIG. 10A, GUI screen 1000A for a guided workflow for purging of one or more components of fluid injector system 104 is illustrated. In embodiments, graphical element 1002A may display an image of a configuration of one or more components, such as an injector head with one or more fluid path components attached, for visual inspection of a setup of fluid injector system 104 in preparation for initiation of a purge process. Graphical element 1002A may include a text message including instructions for conducting a purge sequence (e.g., “Purge procedure set using Auto Purge. Inspect for air. Repeat as necessary.”) for fluid injector system 104. Once a user has determined correct setup of the fluid injector system and one or more components, graphical element 1006A may allow a user to select to proceed to a guided workflow for initiating a purge sequence of fluid injector system 104, for example as illustrated in GUI screen 1000B.

Referring now to FIG. 10B, GUI screen 1000B for a guided workflow for purging of one or more components of fluid injector system 104 is shown. 10B, GUI screen 1000B may include graphical elements 1002B in the form of a warning (e.g. an air embolism hazard warning) to alert the user to ensure that a patient is not connected to fluid injector system 104 during a purge operation. Graphical element 1002B may display a text message (e.g., “Serious injury or harm may occur. Ensure patient is not connected”), in various embodiments including, for example some highlighting process, such as flashing the text message, further detailing a warning associated with the air purging process. Graphical element 1002B may allow a user to select initiate automatic purging of the fluid path elements by fluid injector system 104 and/or to proceed to a subsequent GUI screen of a guided workflow for purging one or more components of fluid injector system 104.

Referring now to FIG. 10C, GUI screen 1000C for a guided workflow for purging of one or more components of fluid injector system 104 is illustrated. Graphical element 1002C may display a configuration of one or more components, such as left syringe and right syringe, of fluid injector system 104 and may provide a status of progress of purging one or more components of fluid injector system 104.

Graphical element 1004C may display a prompt for a user to select to auto-purge one or more components of fluid injector system 104. Alternatively, graphical element 1004C may display a prompt for a user to select to manually purge one or more components of fluid injector system 104. For example, upon receiving a selection of graphical element 1004C for automatic purging, injector management system 102 may provide GUI screen 1000D of a guided workflow for automatically purging one or more components of fluid injector system 104.

In embodiments, graphical element 1004C may allow a user to select to manually purge one or more components of fluid injector system 104, for example to manually remove air from syringe or remove residual air from one or both syringes that remain after an automatic purge sequence. For example, upon receiving a selection of graphical element 1004C for manual purging, injector management system 102 may provide GUI screen 1000E of a guided workflow to allow a user to manually purging one or more components of fluid injector system 104.

In embodiments, after selection of graphical element 1004C for automatic or manual purging, graphical element 1006C may allow a user to select to proceed to a subsequent GUI screen of a guided workflow for purging one or more components of fluid injector system 104, for example based on a user's selection of manual purging or automatic purging.

Referring now to FIG. 10D, GUI screen 1000D, for a guided workflow for automatically purging of one or more components of fluid injector system 104 is illustrated. Graphical element 1002D may display a configuration of one or more components, such as left syringe and right syringe, of fluid injector system 104 setting up for an automatic purge sequence. Graphical element 1002D may provide a status of progress of automatic purging of one or more components of fluid injector system 104.

Referring now to FIG. 10E, GUI screen 1000E for a guided workflow for purging of one or more components of fluid injector system 104 is shown. In embodiments, graphical element 1002E may display a configuration of one or more components, such as left syringe and right syringe, of fluid injector system 104 with graphical icons, for example arrows, for a user to activate to manually purge a syringe. Pressing the graphical icon (arrow) manually advances the plunger and associated piston to purge air from the syringe. The user may monitor the syringe and associated fluid path to determine if and when all air has been manually purged and then stop pressing the arrow to stop the air purge.

Referring now to FIG. 10F, GUI screen 1000F for a guided workflow for manually purging of one or more components of fluid injector system 104 is illustrated. Graphical element 1002F may display a configuration of one or more components, such as left syringe and right syringe, of fluid injector system 104 to indicate to a user that manual purging of one or more components of fluid injector system 104 has been completed. In embodiments, graphical element 1006F may allow a user to select to proceed to a subsequent GUI screen of a guided workflow for purging one or more components of fluid injector system 104.

Referring now to FIG. 10G, GUI screen 1000G for a guided workflow for automatically purging of one or more components of fluid injector system 104 is illustrated. Graphical element 1002G may display a configuration of one or more components, such as left syringe and right syringe, of fluid injector system 104 and may provide a status of progress of purging one or more components of fluid injector system 104. In embodiments, graphical element 1004G may display a prompt for a user to select one or more components of fluid injection system 104 to automatically purge (e.g., left syringe, right syringe, or both left and right syringes). Graphical element 1006G may allow a user to select to proceed to a subsequent GUI screen of a guided workflow for purging one or more components of fluid injector system 104.

Referring now to FIG. 10H, GUI screen 1000H for a guided workflow for purging of one or more components of fluid injector system 104 is shown. Graphical element 1002H may display a configuration of one or more components, such as left syringe and right syringe, of fluid injector system 104 including, for example, a fluid volume after a purge operation of the fluid injector system. Additionally or alternatively, graphical element 1002H may provide a status of progress of purging one or more components of fluid injector system 104.

In embodiments, graphical element 1004H may display a prompt for a user to select to auto-purge or manually purge one or more components of fluid injector system 104. For example, if a user determines that the one or more injector components still contain air, the user may select graphical element 1004H for automatic purging or manually purging the system. Upon selection, injector management system 102 may return to GUI screen 1000D of a guided workflow for automatically purging one or more components of fluid injector system 104 or may return to GUI screen 1000E of a guided workflow for manually purging one or more components of fluid injector system 104. Graphical element 1004H may allow a user to select to proceed to a subsequent GUI screen of a guided workflow for purging one or more components of fluid injector system 104.

Referring now to FIG. 10I, GUI screen 1000I for a guided workflow for purging of one or more components of fluid injector system 104 is shown. Graphical element 1002I may display an image of a configuration of the injector head, of fluid injector system 104 in a configuration for the fluid injection procedure, that is, with the injector head positioned in the downward position. Graphical element 1002I may display a text message (e.g., “SPAT set-up complete. Rotate injector head to downward position”) to instruct the user to place fluid injector system 104 in the appropriate position for the fluid injection procedure. Graphical element 1006I may allow a user to select to proceed to a subsequent GUI screen of a guided workflow for purging one or more components of fluid injector system 104.

Referring now to FIG. 10J, GUI screen 1000J for a guided workflow for preparing of one or more components of fluid injector system 104 for a fluid injection procedure is illustrated. Graphical element 1002J may display an image of a configuration of one or more components, such as an injector head in the downward facing, ready for injection position with one or more components attached. Graphical element 1002J may display a text message (e.g., “Injector set-up complete. Connect tubing to patient”) regarding the configuration of the one or more components of fluid injector system 104.

Graphical element 1006J may allow a user to select to proceed to the next guided workflow, for example a workflow associated with connecting fluid injector system 104 to a patient. For example, upon receiving a selection of graphical element 1006J, injector management system 102 may provide a guided workflow for configuring the fluid injector system to inject the contrast and saline into a patient.

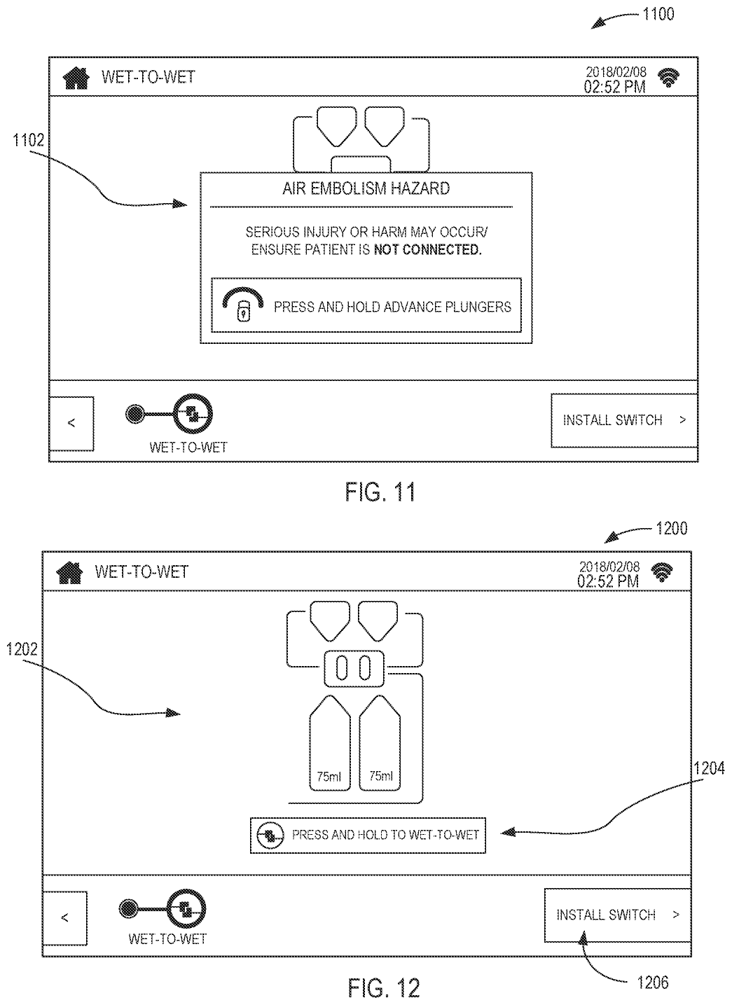

Referring now to FIG. 11, GUI screen 1100 of a guided workflow for connecting fluid injector system 104 with a patient is illustrated. As shown in FIG. 11, GUI screen 1100 may include graphical element 1102, in the form of a warning (e.g. an air embolism hazard warning) when purging a syringe prior to preparing to connect fluid injector system 104 with a circulatory system of a patient. As used herein, the term “wet to wet” means a connection between a fluid filled injector system and the circulatory system of a patients. Graphical element 1102 may provide further details of a potential hazard associated with a wet to wet connection between a fluid injector system and a patient “serious injury or harm may occur, ensure patient is not connected” in FIG. 1100) regarding a warning associated with absence of any air in one or more fluid path components of fluid injector system 104. Graphical element 1102 may allow a user to select to manually purge any remaining air from a fluid path of fluid injector system 104 prior to connecting the system to a patient.

Referring now to FIG. 12, GUI screen 1200 of a guided workflow notification regarding a connection of one or more tubing components of a fluid injector system is illustrated. Graphical element 1202 may display an image of one or more components of fluid injector system 104 during a wet to wet connection protocol. In embodiments, graphical element 1202 may allow a user to confirm that a wet connection of one or more tubing components is made and/or a text message (e.g., “press and hold to Wet-to-Wet” in FIG. 1200) regarding the connection. In embodiments, graphical element 1206 may allow a user to select to proceed to a subsequent GUI screen or a subsequent GUI screen of a guided workflow notification regarding a connection of one or more tubing components of a fluid injector system.

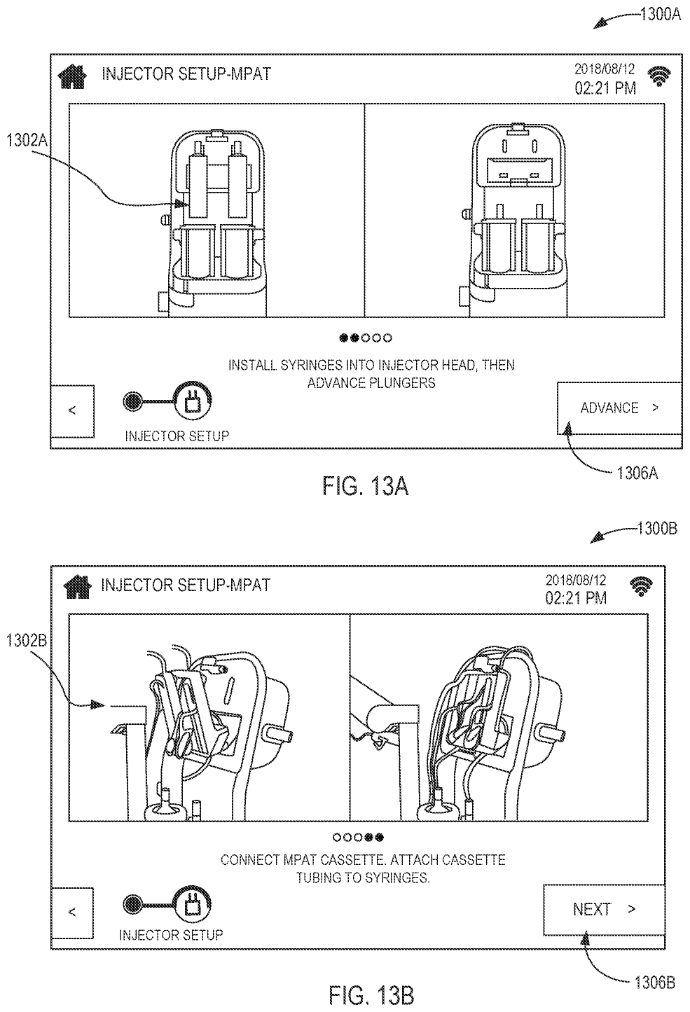

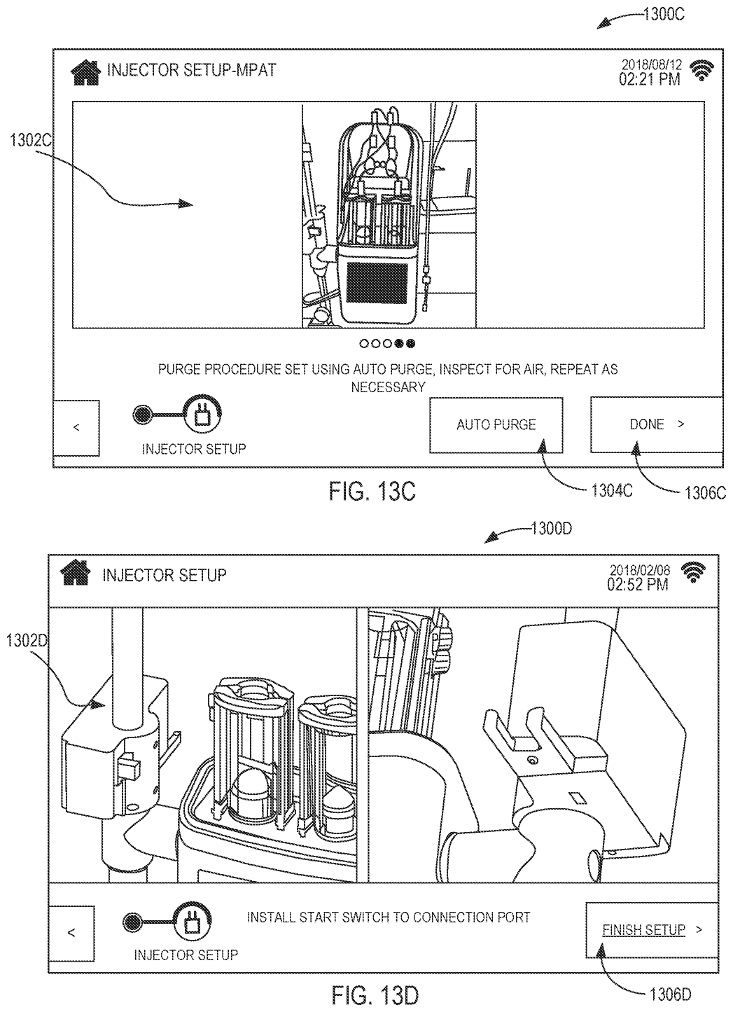

Referring now to FIGS. 13A-13D, GUI screens of a guided workflow for setup of one or more multi-patient disposable components of a fluid injector system is illustrated. The guided workflow shown in FIGS. 13A-13D may follow another guided workflow, such as the guided workflow shown in FIGS. 6A-6B. For example, upon completion of the guided workflow for selection and setup of one or more disposable components of a fluid injection system, injector management system 102 may progress to a guided workflow for setup of one or more disposable components of fluid injector system 104 in FIGS. 13A to 13D).

Referring now to FIG. 13A, GUI screen 1300A for a guided workflow for engagement of two syringes into the pressure jackets of fluid injector system 104 is illustrated. Graphical element 1302A may display an image of one or more syringes and a process for engagement of the one or more syringes with an injector head, of fluid injector system 104. Graphical element 1302A may further display a text message (e.g., “Install syringes into injector head, then ADVANCE PLUNGERS”) regarding an instruction to engage the one or more syringes and associated plungers with the pistons of the fluid injector head. Graphical element 1306A may allow a user to select to proceed to a subsequent GUI screen of a guided workflow for setup of one or more disposable components of fluid injector system 104.

Referring now to FIG. 13B, GUI screen 1300B for a guided workflow for setup of one or more disposable components of a fluid injector system 104 is illustrated. Graphical element 1302B may display an image of a setup configuration of one or more components, such as an MPAT cassette and associated tubing with an injector head of fluid injector system 104. Graphical element 1302B may illustrate the proper connection configuration of the cassette with the injector head. Graphical element 1302B may display a text message (e.g., “Connect MPAT cassette. Attach cassette tubing to syringes”) regarding an instruction to configure the one or more components. In embodiments, graphical element 1306B may allow a user to select to proceed to a subsequent GUI screen of a guided workflow for setup of one or more disposable components of fluid injector system 104.

Referring now to FIG. 13C, GUI screen 1300C illustrates an alternative second GUI screen for a guided workflow for setup and automatic purging air of one or more disposable components of fluid injector system 104. Graphical element 1302C may display an image of a configuration of one or more components, such as a single-patient disposable set (SPAT) cassette, associated tubing engaged with an injector head, of fluid injector system 104 and provide a prompt for a user to automatically prime the system once correctly installed. Graphical element 1302C may display a text message (e.g., “Purge procedure set using Auto Purge. Inspect for air. Repeat as necessary.”) regarding an instruction to perform an automatic purge operation of one or more components of fluid injector system 104. In embodiments, graphical element 1304C may allow a user to select to initiate an automatic purge operation of one or more components of fluid injector system 104. In embodiments, graphical element 1306C may allow a user to select to proceed to a subsequent GUI screen of a guided workflow for setup of one or more disposable components of fluid injector system 104.

Referring now to FIG. 13D, GUI screen 1300D for a guided workflow for setup of one or more disposable components of a fluid injector system is shown. Graphical element 1302D may display an image of a configuration of one or more components, such as an injector head with one or more components attached, of fluid injector system 104. Graphical element 1302D may display a text message (e.g., “Install start switch to connection port” in FIG. 13D) regarding a configuration of one or more components, such as a start switch, of fluid injector system 104. Graphical element 1306D may allow a user to confirm completion of the guided workflow for setup of one or more disposable components of a fluid injector system and/or proceed to another guided workflow.

Referring now to FIG. 14, a GUI screen 1400 that provides a status indication of steps of one or more guided workflows for operation of a fluid injector system 104 is shown, including graphical elements 1402. Graphical elements 1402 may provide a user the ability to determine a status regarding completion of steps of one or more guided workflows associated with setup, operation, and teardown of a configuration of fluid injector system 104 by selecting one or more of graphical elements 1402. In embodiments, graphical elements 1402 may be provided in a plurality of rows, where each row represents an individual guided workflow.

Referring now to FIG. 15, GUI screen 1500 providing information regarding one or more fluid injection protocols that may to be used during a fluid injection procedure is illustrated. GUI screen 1500 may include one or more graphical elements 1502 that may provide specific details (e.g., fluid type, flow rate, volume, duration, rise time, etc.) regarding the one or more fluid injection protocols that are to be used during a fluid injection procedure. GUI screen 1500 may include graphical element 1506 that allows a user to arm fluid injector system 104 to perform a fluid injection procedure.

Referring now to FIG. 16, GUI screen 1600 providing summary information for a fluid injection procedure performed by fluid injector system 104 is shown. GUI screen 1400 may include graphical elements 1402 providing a user the ability to view specific details (e.g., number of injections, fluid volume, duration, notes, user ID, patient information, etc.) in the form of a case summary of one or more fluid injection procedures performed by fluid injector system 104. GUI screen 1600 may include graphical plots of a fluid injection procedure indicating amount of fluid injected over time or other graphical plot. GUI screen 1600 may include graphical element 1606 that allows a user to end a view of a case summary and return to a GUI screen that provides access to additional case summaries or to proceed to the next injection procedure.

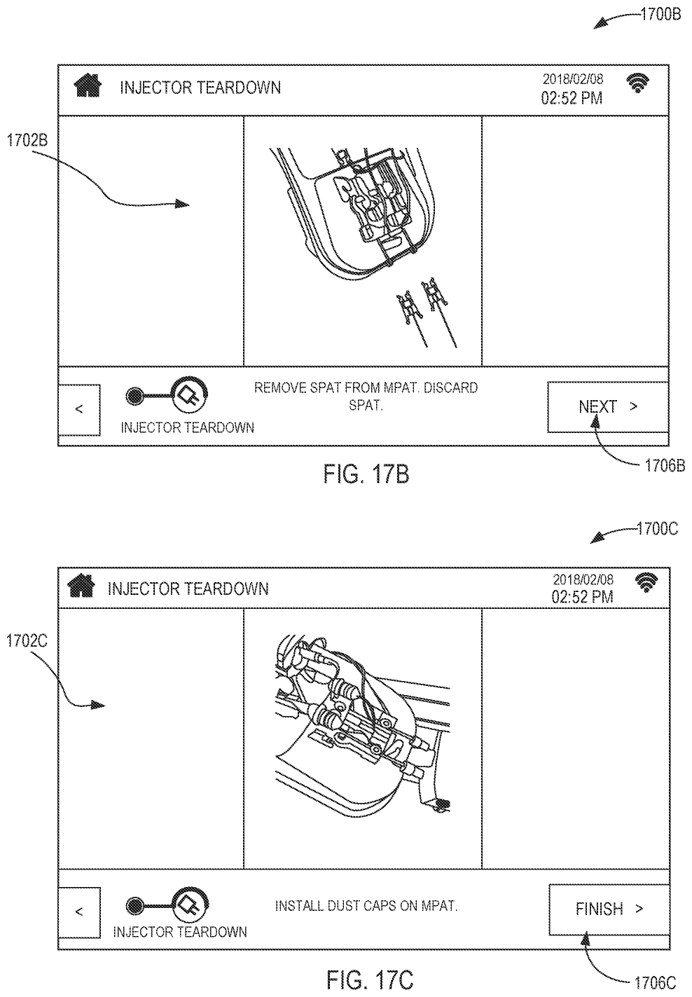

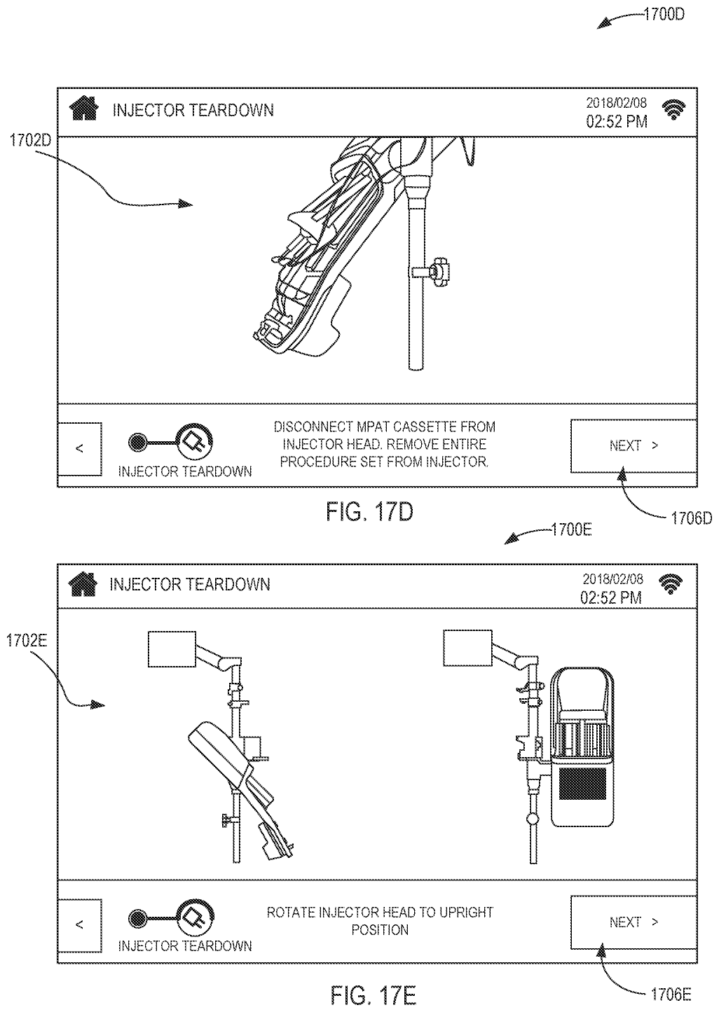

Referring now to FIGS. 17A-17E, GUI screens of a guided workflow for disassembly of one or more components of fluid injector system 104 is illustrated. The guided workflow shown in FIGS. 17A-17E may follow other guided workflows, such as the guided workflow for operation of fluid injector system 104 during a fluid injection procedure as described herein. For example, upon completion of a guided workflow for operation of fluid injector system 104 during a fluid injection procedure, injector management system 102 may progress to a guided workflow for disassembly of one or more components of fluid injector system 104.

Referring now to FIG. 17A, GUI screen 1700A for a guided workflow for disassembly of one or more components of fluid injector system 104 is shown. Graphical element 1702A may display an image of a configuration of one or more components, such an injector head and associated fluid sources and tubing components, of fluid injector system 104 after a fluid injection procedure and may direct a user to an initial step in the teardown procedure, such as removing fluid lines and attached bulk fluid sources. Graphical element 1702A may display a text message (e.g., “Remove fill lines with bulk supplies attached from fill line sensors”) regarding an instruction to remove the one or more components, such as the tubing components, of fluid injector system 104. Graphical element 1702A may include further textual or graphical instructions on the disposal or recycling of one or more of the removed fluid injector components. For example, 1702A may instruct a user to place the fluid line components into a medical waste container and place a glass bulk contrast container in a recycling container associated with glass. In other embodiment, guide workflow of 1702A may instruct a user how to remove any residual volume of contrast agent from the bulk contrast container prior to recycling the container and may further instruct a user how to collect residual contrast agent for recycling or disposal as medical waste. In embodiments, graphical element 1706A may allow a user to select to proceed to a subsequent GUI screen of a guided workflow for disassembly of one or more components of fluid injector system 104.

Referring now to FIG. 17B, GUI screen 1700B for a guided workflow for further disassembly of one or more components, such as a plastic fluid path cassette, of fluid injector system 104. Graphical element 1702B may display an image of one or more components, such as an MPAT and/or SPAT, of fluid injector system 104 and provide guidance for removing the component from fluid injector system 104 for disposal or recycling. Graphical element 1702B may display a text message (e.g., “Remove SPAT from MPAT. Discard SPAT.”) including an instruction to dispose of one or more components of fluid injector system 104. Graphical element 1706B may allow a user to select to proceed to a subsequent GUI screen of a guided workflow for disassembly of one or more components of fluid injector system 104 after completion of the illustrated teardown step.

Referring now to FIG. 17C, GUI screen 1700C for a guided workflow for disassembly of one or more components of fluid injector system 104. Graphical element 1702C may display an image of a configuration of one or more components, such as a cassette associated with an MPAT, of fluid injector system 104. Graphical element 1702C may display a text message (e.g., “Install dust caps on MPAT.”) regarding an instruction to prepare the one or more components of fluid injector system 104 for disposal or recycling, for example by preventing spillage of residual contrast or medical fluid from the component. Graphical element 1706C may allow a user to select to proceed to a subsequent GUI screen of a guided workflow for disassembly of one or more components of fluid injector system 104.

Referring now to FIG. 17D, GUI screen 1700D for a guided workflow for disassembly of a multi-patient (MPAT) set cassette and associated features of fluid injector system 104 is illustrated. Graphical element 1702D may display an image of a configuration of one or more components, such as the MPAT, cassette of fluid injector system 104 including details of its disconnection from the fluid injector head. Graphical element 1702D may display a text message including instructions to disassemble one or more components of fluid injector system 104 (e.g., “Disconnect MPAT cassette from injector head. Remove entire procedure set from injector.”).

Additionally or alternatively, graphical element 1702D may display a text message (e.g., “Are you interested in Recycling Contrast or Plastic”) and allow a user to select a graphical element (e.g., a button indicated for recycle) to receive information regarding recycling one or more portions of the one or more components. In embodiments, graphical element 1702D may provide instructions regarding contrast recycling. Additionally or alternatively, 1702D may instruct a user to recycle one or more portions of the one or more components. In one example, graphical element 1702D may display a text message to “Pull in 25 ml air, tilt head down, dispense into Bayer® Fluid Recycling Container,” and contact a phone number. In some embodiments, graphical element 1702D may provide instructions regarding plastic recycling. Further, graphical element 1702D may display a text message “Remove tubing set, remove rubber cover, remove plunger, separate rubber cover.” In some embodiments graphical element 1702D may provide instructions regarding separating polycarbonate components, such as the plunger, cassette, and syringe. Additionally or alternatively, graphical element 1702D may display a list of recycling resources for respective components. In some embodiments graphical element 1702D may display images or text messages regarding information for identification of components and respective material types (e.g., component image, material type, recycling code). Graphical element 1706D may allow a user to select to proceed to a subsequent GUI screen of a guided workflow for disassembly of one or more components of fluid injector system 104. In some non-limiting embodiments, 1702D may instruct a user to remove fluid tubing from the cassette skeleton and dispose of the tubing and place the cassette skeleton in an appropriate recycling container associated with the type of plastic. In embodiments, GUI screen 1700 may allow a user to confirm recycling of the one or more component to allow tracking of the amount of components that have been recycled.

Referring now to FIG. 17E, GUI screen 1700E for a guided workflow for disassembly of one or more components of fluid injector system 104 illustrates preparing the injector head for installation of the next set of one or more disposable components. Graphical element 1702E may display an image of a configuration of an injector head, of fluid injector system 104 in both the downward facing, injection configuration and the upright preparation position. Graphical element 1702E may display a text message (e.g., “Rotate Injector head to upright position.”) with instructions to configure the injector head of fluid injector system 104 for preparing for the next fluid injection setup. Graphical element 1706E may allow a user to select to proceed to another guided workflow, such as returning to the GUI screens associated with guided workflow installing one or more components and setting up fluid injector system 104 for the next injection of series of injections.

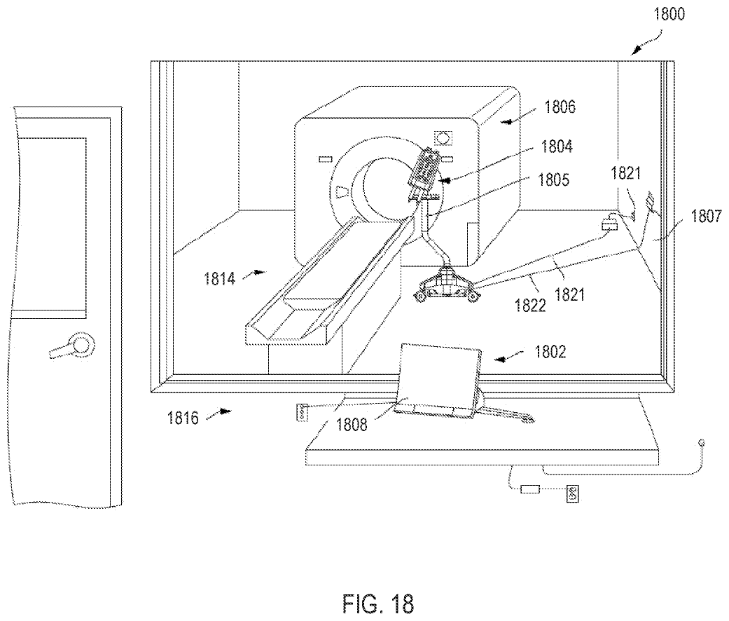

Referring now to FIG. 18, a non-limiting embodiment of medical imaging suite 1800 is illustrated. Medical imaging suite 1800 may be located in hospitals and/or imaging centers for use in performing imaging procedures on patients for the purpose of diagnosing disease and/or other abnormalities. Medical imaging suite 1800 may include scan room 1814 in which fluid injector system 1804 and medical imaging system 1806 are located and control room 1816 from which an individual (e.g., a Radiologist, a Technologist, or other medical personnel) may monitor a patient through one or more workstation devices 1802 associated with fluid injector system 1804 and/or medical imaging system 1806. Fluid injector system 1804 may be the same as or similar to fluid injector system 104. In embodiments, workstation device 1802, which includes display device 1808, may be the same as or similar to workstation device 106 described herein. In embodiments, injector management system 102 may be a component of workstation device 1802. As shown in FIG. 18, workstation device 1802 may be used (e.g., by medical personnel) to set up and/or execute a fluid injection procedure for a patient and may include one or more of the guided workflow processes described herein.

Although the above systems, methods, and computer program products have been described in detail for the purpose of illustration based on what is currently considered to be the most practical and preferred embodiments, it is to be understood that such detail is solely for that purpose and that the present disclosure is not limited to the described embodiments or aspects but, on the contrary, is intended to cover modifications and equivalent arrangements that are within the spirit and scope of the appended claims. For example, it is to be understood that the present disclosure contemplates that, to the extent possible, at least one feature of any embodiment or aspect can be combined with at least one feature of any other embodiment.

1. A method for providing one or more guided workflow features on a fluid injector system, comprising:
receiving, with at least one processor, data associated with the fluid injector system;
displaying, with the at least one processor, a first graphical user interface (GUI) screen associated with a guided workflow for operation of the fluid injector system on one or more display devices based on the data associated with one or more components of the fluid injector system;
receiving, with the at least one processor, a first user input via a first graphical element of the first GUI screen or an input device;
displaying, with the at least one processor, a second GUI screen associated with the guided workflow for operation of the fluid injector system on the one or more display devices based on receiving the first user input;
executing, with the at least one processor, a first operation of the fluid injector system based on the first user input; and
receiving, with the at least one processor, a second user input via a second graphical element of the second GUI screen or the input device;
wherein the second GUI screen provides information that is sequential to the first GUI screen regarding the guided workflow for operation of the fluid injector system.

2. The method of claim 1, further comprising:
executing a second operation of the fluid injector system based on the second user input.

3. The method of claim 1, wherein displaying the first GUI screen on the one or more display devices comprises:
displaying the first GUI screen on one or more display devices of the fluid injector system.

4. The method of claim 1, wherein displaying the first GUI screen on the one or more display devices comprises:
displaying at least a first portion of the first GUI screen on a first display device of the fluid injector system; and
displaying at least a second portion of the first GUI screen on a second display device that is independent of the fluid injector system.

5. The method of claim 1, wherein displaying the second GUI screen on the one or more display devices comprises:
displaying at least a first portion of the second GUI screen on a first display device of the fluid injector system based on receiving the first user input via the graphical element of the first GUI screen or the input device; and
displaying at least a second portion of the second GUI screen on a second display device that is independent of the fluid injector system based on receiving the first user input via the graphical element of the first GUI screen or the input device.

6. The method of claim 1, wherein displaying the first GUI screen on the one or more display devices comprises:
displaying the first GUI screen on a display device of a workstation device that is independent of the fluid injector system.

7. The method of claim 1, wherein receiving data associated with the fluid injector system comprises:
receiving data associated with one or more disposable components of the fluid injector system from a reader device that reads at least one barcode associated with the one or more disposable components of the fluid injector system;
wherein the method further comprising:
displaying on the one or more display devices, a first GUI screen associated with a guided workflow for disposal and recycling of one or more components used during a fluid injection procedure of the fluid injector system based on the data associated with the one or more disposable components of the fluid injector system;
receiving a third user input via a graphical element of the first GUI screen associated with a guided workflow for recycling of one or more components used during the fluid injection procedure of the fluid injector system or the input device, wherein the third user input comprises an indication that the one or more disposable components of the fluid injector system are to be recycled;
displaying on the one or more display devices, a second GUI screen associated with the guided workflow for disposal and recycling of the one or more components based on the receiving the third user input, wherein the second GUI screen comprises at least one instruction for a procedure for recycling the one or more disposable components of the fluid injector system; and
displaying on the one or more display devices, a third GUI screen associated with the guided workflow for disposal and recycling of the one or more components, wherein the second GUI screen comprises data associated with a sustainability score indicating an environmental sustainability of a user.

8. A system for providing one or more guided workflow features on a fluid injector system, comprising:
at least one processor programmed or configured to:
receive data associated with the fluid injector system;
display a first graphical user interface (GUI) screen associated with a guided workflow for operation of the fluid injector system on one or more display devices based on the data associated with one or more components of the fluid injector system;
receive a first user input via a first graphical element of the first GUI screen or an input device;
display a second GUI screen associated with the guided workflow for operation of the fluid injector system on the one or more display devices based on receiving the first user input;
execute a first operation of the fluid injector system based on the first user input; and
receive a second user input via a second graphical element of the second GUI screen or the input device;
wherein the second GUI screen provides information that is sequential to the first GUI screen regarding the guided workflow for operation of the fluid injector system.

9. The system of claim 8, wherein the at least one processor is further programmed or configured to:
execute a second operation of the fluid injector system based on the second user input.

10. The system of claim 8, wherein, when displaying the first GUI screen on the one or more display devices, the at least one processor is programmed or configured to:
display the first GUI screen on one or more display devices of the fluid injector system.

11. The system of claim 8, wherein, when displaying the first GUI screen on the one or more display devices, the at least one processor is programmed or configured to:
display at least a first portion of the first GUI screen on a first display device of the fluid injector system; and
display at least a second portion of the first GUI screen on a second display device that is independent of the fluid injector system.

12. The system of claim 8, wherein the at least one processor is further programmed or configured to:
display at least a first portion of the second GUI screen on a first display device of the fluid injector system based on receiving a user input via a graphical element of the first GUI screen or the input device; and
display at least a second portion of the second GUI screen on a second display device that is independent of the fluid injector system based on receiving the user input via the graphical element of the first GUI screen or the input device.

13. The system of claim 8, wherein, when displaying the first GUI screen on the one or more display devices, the at least one processor is programmed or configured to:
display the first GUI screen on a display device of a workstation device that is independent of the fluid injector system.

14. The system of claim 8, wherein, when receiving data associated with the fluid injector system, the at least one processor is programmed or configured to:
receive data associated with one or more disposable components of the fluid injector system from a reader device that reads at least one barcode associated with the one or more disposable components of the fluid injector system;
wherein the at least one processor is further programmed or configured to:
display on the one or more display devices, a first GUI screen associated with a guided workflow for disposal and recycling of one or more components used during a fluid injection procedure of the fluid injector system based on the data associated with the one or more disposable components of the fluid injector system;
receive a third user input via a graphical element of the first GUI screen associated with a guided workflow for recycling of one or more components used during the fluid injection procedure of the fluid injector system or the input device, wherein the third user input comprises an indication that the one or more disposable components of the fluid injector system are to be recycled;
display on the one or more display devices, a second GUI screen associated with the guided workflow for disposal and recycling of the one or more components based on the receiving the third user input, wherein the second GUI screen comprises at least one instruction for a procedure for recycling the one or more disposable components of the fluid injector system; and
display on the one or more display devices, a third GUI screen associated with the guided workflow for disposal and recycling of the one or more components, wherein the second GUI screen comprises data associated with a sustainability score indicating an environmental sustainability of a user.

15. A computer program product for providing one or more guided workflow features on a fluid injector system, the computer program product comprising at least one non-transitory computer-readable medium including one or more instructions that, when executed by at least one processor, cause the at least one processor to:
receive data associated with the fluid injector system;
display a first graphical user interface (GUI) screen associated with a guided workflow for operation of the fluid injector system on one or more display devices based on the data associated with one or more components of the fluid injector system;
receive a first user input via a first graphical element of the first GUI screen or an input device;
display a second GUI screen associated with the guided workflow for operation of the fluid injector system on the one or more display devices based on receiving the first user input;
execute a first operation of the fluid injector system based on the first user input; and
receive a second user input via a second graphical element of the second GUI screen or the input device;
wherein the second GUI screen provides information that is sequential to the first GUI screen regarding the guided workflow for operation of the fluid injector system.

16. The computer program product of claim 15, wherein the one or more instructions further cause the at least one processor to:
execute a second operation of the fluid injector system based on the second user input.

17. The computer program product of claim 15, wherein the one or more instructions that cause the at least one processor to display the first GUI screen on the one or more display devices, cause the at least one processor to:
display the first GUI screen on one or more display devices of a fluid injector system.

18. The computer program product of claim 15, wherein the one or more instructions further cause the at least one processor to:
display at least a first portion of the first GUI screen on a first display device of a fluid injector system; and
display at least a second portion of the first GUI screen on a second display device that is independent of the fluid injector system.

19. The computer program product of claim 15, wherein the one or more instructions that cause the at least one processor to display the first GUI screen on the one or more display devices, cause the at least one processor to:
display at least a first portion of the second GUI screen on a first display device of the fluid injector system based on receiving the user input via the graphical element of the first GUI screen or the input device; and
display at least a second portion of the second GUI screen on a second display device that is independent of the fluid injector system based on receiving the user input via the graphical element of the first GUI screen or the input device.

20. The computer program product of claim 15, wherein the one or more instructions that cause the at least one processor to receive data associated with the fluid injector system, cause the at least one processor to:
receive data associated with one or more components of the fluid injector system from a reader device that read a barcode associated with the one or more components of the fluid injector system;
wherein the one or more instructions further cause the at least one processor to:
display on the one or more display devices, a first GUI screen associated with a guided workflow for disposal and recycling of one or more components used during a fluid injection procedure of the fluid injector system based on the data associated with the one or more disposable components of the fluid injector system;
receive a third user input via a graphical element of the first GUI screen associated with a guided workflow for recycling of one or more components used during the fluid injection procedure of the fluid injector system or the input device, wherein the third user input comprises an indication that the one or more disposable components of the fluid injector system are to be recycled;
display on the one or more display devices, a second GUI screen associated with the guided workflow for disposal and recycling of the one or more components based on the receiving the third user input, wherein the second GUI screen comprises at least one instruction for a procedure for recycling the one or more disposable components of the fluid injector system; and
display on the one or more display devices, a third GUI screen associated with the guided workflow for disposal and recycling of the one or more components, wherein the second GUI screen comprises data associated with a sustainability score indicating an environmental sustainability of a user.
